(19) United States
(12) Patent Application Publication
(10) Pub. No.: US 2022/0344000 A1
(43) Pub. Date: Oct. 27, 2022

(54) METHOD OF PREDICTING PRODUCTION STABILITY OF CLONAL CELL LINES

(71) Applicants: GLAXOSMITHKLINE INTELLECTUAL PROPERTY DEVELOPMENT LIMITED, Brentford (GB); UCL Business LTD, London (GB)

(72) Inventors: Alexander HENDERSON, Stevenage (GB); Nicola RICHMOND, Brentford (GB); Shahla SALEHI, Stevenage (GB); Remben TALABAN, Stevenage (GB)

(21) Appl. No.: 17/763,998

(22) PCT Filed: Sep. 25, 2020
(86) PCT No.: PCT/EP2020/076831

§ 371 Date: Mar. 25, 2022

Related U.S. Application Data
(60) Provisional application No. 62/906,798, filed on Sep. 27, 2019.

(51) Int. Cl. G16B 20/10; C12N 5/00

(52) U.S. Cl. CPC G16B 20/10; C12N 5/0018

(57) ABSTRACT

The invention relates to a method of predicting production stability and/or production instability of a clonal cell line, the method comprising the steps of a) growing two or more clonal cell lines in separate cell cultures b) karyotyping the cells in each cell culture; and c) deriving a genomic instability value from the karyotyping of step (b). The invention also relates to methods of selecting a cell line which expresses a therapeutic protein and method of selecting a high-titre producing clonal cell line for large scale therapeutic protein production.

### FIELD OF THE INVENTION

The invention generally relates to methods of developing cell lines for therapeutic protein production, particularly methods of predicting production stability and/or production instability of a clonal cell line. The invention also relates to methods of selecting a cell line which expresses a therapeutic protein and methods for selecting a high titre producing clone for large scale therapeutic protein production.

### BACKGROUND TO THE INVENTION

Mammalian cell lines are used for production of recombinant therapeutic proteins. Examples of such mammalian cell lines include murine myeloma cells (NS0), baby hamster kidney cells (BHK), human embryonic kidney cells (HEK-293) and Chinese hamster ovary cells (CHO), with over 80% of currently approved recombinant proteins are expressed in CHO platforms (Butler & Spearman, 2014; Walsh, 2018). The success of CHO cell lines as a platform can be largely attributed to their ability to be cultured at high densities, their ease of exogenous DNA uptake and relative ease of adaptation to serum free suspension culture.

A major bottleneck in the process for producing therapeutic proteins using mammalian cells is the time taken to isolate a clonal cell line with production stability. Production stability assessments across the industry can vary between 60 to >100 generations (BioPhorum Development Group, Stability Survey 2018) with a substantial number of cell lines requiring to be assessed to account for a large proportion of the cells being productionally unstable. Without maintenance of production titre throughout the manufacturing period, process yield can have a significant impact on timelines as manufacturing schedules are typically booked up to at least a year in advance. As such, unexpectedly low production titres can lead to repeat manufacture runs having an enormous impact on scheduling and a knock-on effect on product distribution.

Accordingly, there is a need in the art for methods of reducing the time taken to identify clonal cell lines with production stability.

### SUMMARY OF THE INVENTION

According to one aspect of the invention, there is provided a method of predicting production stability and/or production instability of a clonal cell line, the method comprising the steps of
(a) growing two or more clonal cell lines in separate cell cultures
(b) karyotyping the cells in each cell culture; and
(c) deriving a genomic instability value from the karyotyping of step (b).

In a further aspect of the invention, there is provided a method of selecting a cell line which expresses a therapeutic protein, the method comprising the steps of
(a) growing two or more clonal cell lines in separate cell cultures
(b) karyotyping the cells in each cell culture
(c) deriving a genomic instability value from the karyotyping of step (b); and
(d) selecting a clonal cell line based on the genomic instability value of step (c).

In yet another aspect of the invention, there is provided a method of selecting a high-titre producing clonal cell line for large scale therapeutic protein production, the method comprising the steps of
(a) growing two or more clonal cell lines in separate cell cultures
(b) karyotyping the cells in each cell culture
(c) deriving a genomic instability value from the karyotyping of step (b); and
(d) selecting a clonal cell line based on the genomic instability value of step (c).

In one embodiment, karyotyping comprises identifying chromosomal aberrations of the clonal cell lines. In another embodiment, karyotyping comprises performing multi-colour fluorescence in situ hybridisation (MFISH), spectral karyotyping (SKY) or Giesma banding (G banding).

In a further embodiment, the methods further comprise after step (b), the step of determining subpopulations of each cell culture by karyotype.

In some embodiments, deriving the genomic instability value comprises assigning each subpopulation as comprising clonal chromosomal aberration (CCA) or non-clonal chromosomal aberration (NCCA). In one embodiment, deriving the genomic value further comprises the step of determining a percentage CCA and/or percentage NCCA for each clonal cell line.

In some embodiments, deriving the genomic instability value comprises determining an average matching cost distribution. In some embodiments, deriving the genomic instability value comprises determining a variance of the average matching cost distribution. In some embodiments, the genomic instability values are used to i) rank the clonal cells by % CCA or variance of the average matching cost distribution; (ii) derive a % CCA threshold or variance of the average matching cost distribution threshold; and (iii) derive a quartile threshold. In one embodiment the genomic instability values are used to derive a % CCA threshold. In one embodiment the % CCA threshold is at least 70%. In one embodiment, the % CCA threshold is 78%.

In some embodiments, the step of karyotyping the cells in each cell culture and/or the step of deriving a genomic instability value from the karyotyping is/are automated. In one embodiment, automation is computer-implemented automation.

In some embodiments, the step of karyotyping the cells in each cell culture is carried out between 10 generations and 40 generations. In some embodiments, the step of karyotyping the cells in each cell culture is carried out after 10, 15 or 20 generations.

In one embodiment, the clonal cell line is a mammalian cell line. In one embodiment, the mammalian cell line is a Chinese Hamster Ovary (CHO) cell line. In one embodiment, the CHO cell line is CHO-K1. In some embodiments, the CHO cell line is a glutamine synthetase (GS) knocked out cell.

### DESCRIPTION OF DRAWINGS/FIGURES

FIGS. 1A-E. A) Population pie charts of each cell line divided into stability and time point categories. CCA (speckled) and NCCA (plain) pie segments highlight an increase in NCCA populations when comparing stable to unstable and early to late. B) Overall CCA and NCCA frequencies were calculated across each stability group and differences between each group was statistically significant (Two-way ANOVA, P=0.01). The grand mean was calculated at 78% indicating a potential threshold for production stability designation. C) CCA and NCCA population frequency difference between early and late time points are statistically significant (Two-way ANOVA, P=<0.0001), indicating that NCCA populations increase over prolonged periods of cell culture, leading to more heterogeneity. The triangles represent the population mean and 95% confidence intervals, blue lines indicate standard deviation. D) Mutations categorised by chromosome; cell lines are represented by the different pattern segments. Chromosome 6 and 8 retain the most mutations with chromosome 6 being mutated in 11 out of 14 cell lines. E) similar bar chart as D except sorted by stability. All chromosomes except 2, 17, 18, and 19 obtained mutations in both stable and unstable cell lines. No pattern of specific chromosome mutations was observed.

FIGS. 2A-D. Three different prediction methods were devised before the unblinding of cell lines after analysing results. Cell lines were sorted by CCA % from high to low and the different prediction methods were applied and the prediction success rate calculated. A) Top and bottom 25%, utilised to identify the most stable and unstable cell lines. B) Threshold prediction based on initial productionally stable and unstable cell line panel; threshold set at CCA 78%. CCA≥78% is considered a productionally stable cell line, conversely <78% is considered as a productionally unstable cell line. C) Cell lines sorted by percent (%) CCA are divided into quartiles to identify top 25% and bottom 50% for cell line triaging. D) Comparison of % CCA and % NCCA in productionally stable and unstable groups (pooled T-test, P=<0.0001).

FIGS. 3A-C. A) CCA (speckled) and NCCA (plain) populations of productionally stable and unstable cell lines that have been sampled on day 8 during a production run. Day 0 time point reflects the cell lines' baseline heterogeneity before entering the production run environment. Increases in NCCA populations was observed after 8 days within the production environment. Day 8 gH2AX represents the same cell lines that have been treated with 1 ng/ml Neocarzinostatin for the duration of the production run. Addition of Neocarzinostatin has increased NCCA populations further (red segments). B) % CCA and % NCCA of stable cell lines across day 0, day 8 and day8 gH2AX (treated with Neocarzinostatin). Stable cell lines obtained a decrease in CCA populations after 8 days within a production run environment (two-way ANOVA, Hochberg's adjusted P-value, P=<0.001***). CCA population decrease was exacerbated further, compared to day 0 and day 8, due to the addition of DNA damaging agent (P=<0.0001*** and P=<0.01**, respectively). C) % CCA and % NCCA of unstable cell lines across day 0, day 8 and day 8 gH2AX. % CCA decreased by 17.5% between day 0 and day 8, however this was insignificant (P=0.07n.$). CCA populations decreased in the presence of Neocarzinostatin leading to ˜40% decrease compared to day 0 (P=<0.0001***) and ˜23% decrease compared to day 8 (P=0.015*).

FIG. 4. A1 and A2) Automated image segmentation using U-Net model. Faithful segmentation of chromosomes allows for robust pseudo colouring using a gaussian mixture model (B1 and B2). C1 and C2) Pairwise linear assignment of chromosomes, together with associated matching cost. A translocation of 10 and 19 is detectable by the algorithm via a large matching cost.

FIGS. 5A-C. A) Comparison of manual and automated (APW) calculated CCA and NCCA subpopulations showing similar profiles in CCA and NCCA proportions within each cell line. B) Comparison of % CCA and % NCCA generated by the automated prediction workflow showing a distinct separation between stable and unstable cell lines, as observed in manual analyses (P=<0.05). C) Dot plot depicting correlation between cell line average cost matching distribution variance and % NCCA indicating variance of average matching cost distribution could be used as a computational biomarker of genetic instability (Large variance=increased variability of matching costs=greater number of mutations).

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference in their entirety.

The term “comprising” encompasses “including” or “consisting” e.g. a composition “comprising” X may consist exclusively of X or may include something additional e.g. X+Y.

The term “consisting essentially of” limits the scope of the feature to the specified materials or steps and those that do not materially affect the basic characteristic(s) of the claimed feature.

The term “consisting of” excludes the presence of any additional component(s).

The term “about” in relation to a numerical value x means, for example, x±10%, 5%, 2% or 1%.

The term “clonal cell line” as used herein refers to a host cell, comprising a gene of interest, that has been single cell sorted. A clonal cell line may undergo a therapeutic protein production stability assessment as described herein, during which the single cell sorted clonal cell line will be grown in a cell culture. Cells grown in said cell culture will share a common ancestry to the respective clonal cell line. It is to be understood that where “two or more clonal cell lines” used, this refers to clonal cell lines that express the same therapeutic protein of interest.

The term “karyotype” as used herein refers to a collection of chromosomes in a cell. The term may also refer to an image of a cell's chromosomes. The karyotype may be used to analyse or determine a cell's chromosomal make up (i.e. karyotyping), for example analysing or determining chromosomal aberrations.

The term “chromosomal aberration” as used herein refers to abnormalities involving the structure or number of chromosomes. Examples of chromosomal aberrations include translocation, deletion, duplication and inversion. A clonal population of cells may be divided into subpopulations of cells comprising the same or similar chromosomal aberration.

The term “clonal chromosomal aberration” as used herein is a chromosomal aberration which is detected at least twice within 20 to 40 randomly examined mitotic figures within a clonal population of cells.

The term “non-clonal chromosomal aberration” as used herein is a chromosomal aberration which is detected in only a single cell within 20 to 40 randomly examined mitotic figures within a clonal population of cells.

The term “genomic instability metric” as used herein refers to a metric by which the level of chromosomal aberrations within the genome of a cellular lineage may be assessed. In other words, the genomic instability metric is the metric by which the karyotypic heterogeneity of a clonal population may be measured. The “genomic instability value” is derived by applying the genomic instability metric to the karyotypes of the cells grown from a clonal cell line.

The term “production stability” as used herein refers to the stability of production of therapeutic protein by a clonal cell line, that is to say production of a consistent titre of therapeutic protein over 4 to 6 months. In some examples, consistent titre is defined as <30% drop in therapeutic protein.

The term “early time point” as used herein refers to the early time point at which samples of the cells are taken to determine their karyotype. This is taken to be between around 10 to 20 generations.

The term “late time point” as used herein refers to the late time point at which samples of the cells are taken to determine their karyotype. This is taken to be between around 80 to 150 generations.

Host cell lines are used as mammalian cell factories to create therapeutic protein producing clonal cell lines. Taking an antibody as an example of a therapeutic protein, a nucleic acid sequence encoding the antibody is cloned into an expression vector and subsequently transfected into the host cell line. Transfected pools are bulked, single cell sorted, and outgrowth of these single cell sorted, clonal cell lines are then assessed for their antibody production (IgG titre). Clonal cell lines are ranked based on their titre and undergo a series of triage events until around 50 clonal cell lines are selected to enter a production stability assessment.

Production stability assessment of a clonal cell line is essential. In order for a clonal cell line to progress to the manufacturing stage, it must produce a consistent amount of therapeutic protein across the manufacturing window (typically 4 to 6 months). A standard production stability assessment involves culturing the clonal cell lines in vessels such as deep well plates, shake flasks or mini-bioreactors across a 4 to 6 month period to reflect the length of time of the manufacturing window. To calculate production stability, maximum titre reads are taken at different timepoints and percent titre change across the time series is calculated. Generally, clonal cell lines which are able to maintain their protein expression to within 30% of their original peak titre during the stability assessment are considered stable (BioPhorum Survey, 2018).

Although several different host cell lines have gained regulatory approval, including murine myeloma (NS0) and human embryo kidney (HEK-293), 80% of mammalian cell culture processes for biopharmaceutical production utilise Chinese hamster ovary (CHO) suspension cells (Walsh, 2018; Wurm, 2004). CHO cells are preferred when expressing therapeutic proteins due to the conservation of mammalian post-translational modifications, which are crucial for mAb-FcγR interactions. Improper post-translational modifications can result in unwanted effects such as altered protein stability, lowered affinity towards a targeted antigen, aberrant clearance rate and immunogenicity profiles. Additionally, the strong track record of CHO as a biologic factory with regulators allows for a smoother approval process (Walsh, 2018).

Several studies have highlighted the karyotypic heterogeneity of the CHOK1 line, indicating a highly mutational environment. Work by Deavan and Peterson (Deaven and Petersen, 1973) highlighted that 24% of their cells contained a chromosome number that differed from the expected 22 (chromosome numbers ranged from 19-23) and the phenomenon still persists to this day (Auer et al., 2018; Vcelar et al., 2018a; Vcelar et al., 2018b; Yusufi et al., 2017).

During a pharmaceutical CHO cell lifecycle, CHO cells undergo constant genomic modifications, which have been shown to attribute to phenotypic differences in clonal cell lines (Derouazi et al., 2006). In addition to the natural mutational tendency of CHOK1 cell lines, the use of the methotrexate (MTX) or methionine sulfoximine (MSX) selection systems have been shown to also compound mutagenesis. A high frequency of chromosomal disturbances such as breakages, dicentric chromosomes and disruption to telomeric structures have also been well documented in human, mouse and hamster cell lines.

In an industry setting, for each therapeutic protein, around 50 clonal cell lines are typically progressed to production stability assessment, from which a single clonal cell line, that is deemed manufacturable, will be selected.

The inventors have identified a correlation between genetic stability/instability within a clonal population of cells and production stability/instability and methods of measuring and analysing the genetic stability/instability to predict production stability/instability of the respective clonal cell line. By applying the methods during cell line development, specifically at an early stage during the 4 to 6 month period for assessing production stability of clonal cell lines, it is possible to triage clonal cell lines predicted to be productionally unstable earlier during cell line development (CLD), thereby increasing CLD capacity and reducing chemistry, manufacturing and controls (CMC) timelines.

Therefore, according to one aspect of the invention, there is provided a method of predicting production stability and/or production instability of a clonal cell line, the method comprising the steps of
(a) growing two or more clonal cell lines in separate cell cultures
(b) karyotyping the cells in each cell culture; and
(c) deriving a genomic instability value from the karyotyping of step (b).

In a further aspect of the invention, there is provided method of selecting a cell line which expresses a therapeutic protein, the method comprising the steps of
(a) growing two or more clonal cell lines in separate cell cultures
(b) karyotyping the cells in each cell culture
(c) deriving a genomic instability value from the karyotyping of step (b); and
(d) selecting a clonal cell line based on the genomic instability value of step (c).

In yet another aspect of the invention, there is provided a method of selecting a high-titre producing clonal cell line for large scale therapeutic protein production, the method comprising the steps of
(a) growing two or more clonal cell lines in separate cell cultures
(b) karyotyping the cells in each cell culture
(c) deriving a genomic instability value from the karyotyping of step (b); and
(d) selecting a clonal cell line based on the genomic instability value of step (c).

In one embodiment, the method is for predicting production instability of a clonal cell line. In one embodiment, the method is for predicting production stability of a clonal cell line.

In one embodiment, the method of predicting production stability of a clonal cell line further comprises a step of identifying a clonal cell line predicted to have production stability based on the genomic instability value of step (c).

In one embodiment, the method of predicting production stability of a clonal cell line further comprises a step of selecting a clonal cell line predicted to have production stability based on the genomic instability value of step (c) for continued cell line development.

In one embodiment, the method of predicting production instability of a clonal cell line further comprises a step of identifying a clonal cell line predicted to have production instability based on the genomic instability value of step (c).

In one embodiment, the method of predicting production instability of a clonal cell line further comprises a step of triaging a clonal cell line predicted to have production instability from cell line development, based on the genomic instability value of step (c).

In one embodiment, there is a method of selecting a cell line which expresses a therapeutic protein, the method comprising the steps of
(a) growing two or more clonal cell lines in separate cell cultures
(b) karyotyping the cells in each cell culture
(c) deriving a genomic instability value from the karyotyping of step (b); and
(d) triaging a clonal cell line based on the genomic instability value of step (c).

In one embodiment, there is a method of selecting a high-titre producing clonal cell line for large scale therapeutic protein production, the method comprising the steps of
(a) growing two or more clonal cell lines in separate cell cultures
(b) karyotyping the cells in each cell culture
(c) deriving a genomic instability value from the karyotyping of step (b); and
(d) triaging a clonal cell line based on the genomic instability value of step (c).

The genomic instability value is used to identify or predict production stability and/or production instability of a clonal cell line. In some embodiments, the genomic instability value is used to identify or predict production instability of the clonal cell line. In one embodiment, the genomic instability value is used to identify or predict production stability of the clonal cell line.

The genetic stability/instability of a clonal cell line may be assessed by analysing the karyotype of the clonal cell line and deriving a genetic instability value from the karyotype analysis.

Karyotype is the chromosomal make-up or characteristics of a cell and karyotyping is the process of analysing the chromosomes (cytogenetics) of the cell to obtain genome-wide characteristics of a cell. The karyotype of a cell is typically analysed by obtaining an image of the cell's chromosomes. Karyotyping may be used for the detection of chromosome instability, for example chromosomal aberrations. Chromosomal aberrations are abnormalities involving the structure or number of chromosomes. Examples of chromosomal aberrations include translocation, deletion, duplication and inversion.

In the present invention, the genetic stability/instability of a clonal cell line is determined by obtaining a clonal population by growing the clonal cell line in a cell culture and assessing, by karyotyping, the chromosomal aberrations within the clonal population, which have spontaneously formed under continuous cell culture.

Therefore, in one embodiment, karyotyping comprises identifying chromosomal aberrations of a clonal cell line. In one embodiment, karyotyping comprises identifying the chromosomal aberrations within a clonal population of cells.

In one embodiment, karyotyping the cells in each cell culture (i.e. clonal population) comprises karyotyping 20 or more, 30 or more, 40 or more, 50 or more, 60 or more, 70 or more, 80 or more, 90 or more or 100 or more cells. In one embodiment, karyotyping the cells in each cell culture comprises karyotyping between 20 to 100 cells. In one embodiment, karyotyping the cells in each cell culture comprises karyotyping 20, 30, 40, 50, 60, 70, 80, 90 or 100 cells. In one embodiment, karyotyping the cells in each cell culture comprises karyotyping 20 cells. In one embodiment, karyotyping the cells in each cell culture comprises karyotyping 30 cells. In one embodiment, karyotyping the cells in each cell culture comprises karyotyping 40 cells. In one embodiment, karyotyping the cells in each cell culture comprises karyotyping 50 cells. In one embodiment, karyotyping the cells in each cell culture comprises karyotyping 60 cells. In one embodiment, karyotyping the cells in each cell culture comprises karyotyping 70 cells. In one embodiment, karyotyping the cells in each cell culture comprises karyotyping 80 cells. In one embodiment, karyotyping the cells in each cell culture comprises karyotyping 90 cells. In one embodiment, karyotyping the cells in each cell culture comprises karyotyping 100 cells. In one embodiment, the step of karyotyping the cells in each cell culture is carried out at an early time point in the production stability assessment. In one embodiment, the step of karyotyping the cells grown from a clonal cell line, that is to say a clonal population, is carried out between 10 to 20 generations of cell growth. In one embodiment, the step of karyotyping is carried out between 15 to 40 generations of cell growth. In one embodiment, the step of karyotyping is carried out after 10 generations or more, 15 generations or more, 20 generations or more, 25 generations or more, 30 generations or more, 35 generations or more or 40 generations or more of cell growth. In one embodiment, karyotyping is carried out after 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 generations of cell growth. In one embodiment, karyotyping is carried out after 10 generations of cell growth. In one embodiment, karyotyping is carried out after 15 generations of cell growth. In one embodiment, karyotyping is carried out after 20 generations of cell growth. In one embodiment, the step of karyotyping is carried out after about 1 month from inoculation of the cell culture media with a clonal cell line. In one embodiment, the step of karyotyping is carried out after 5 passages, 10 passages, 15 passages, 20 passages, 25 passages, 40 passages, or 35 passages. In one embodiment, the step of karyotyping is carried out after 6 passages. In one embodiment, the step of karyotyping is carried out after about 7 passages. In one embodiment, the step of karyotyping is carried out after 10 passages.

Karyotyping is typically carried out using mitotic cells that have been arrested during metaphase, when chromosomes are most condensed and, therefore, more clearly visible. A person skilled in the art will be familiar with chromosome isolation techniques, such as disrupting the spindle fibres by incubation with colcemid or colchicine to prevent the cells from proceeding to the subsequent anaphase stage, treating with a hypotonic solution and preserving the cells in their swollen state with Carnoy's fixative before fixing onto slides for analysis. A skilled person would also be familiar with methods for carrying out the chromosome staining techniques.

Chromosome staining techniques are well known in the art. For example, chromosome staining techniques such as Giesma banding (G banding), multi-colour fluorescence in situ hybridisation (MFISH), comparative genomic hybridisation (CGH) and spectral karyotyping (SKY) allow for effective karyotyping, including analysis of chromosomal aberrations. With G banding, metaphase chromosomes are pre-treated with a protease such as trypsin and stained with Giesma stain. Giesma is a visible light dye that binds to DNA through intercalation. MFISH is a technique, utilising species and chromosomal-specific sequences conjugated to different fluorophores, that enables the combination of multiple colours to produce karyotype images of ‘painted’ chromosomes after hybridisation. Painting of chromosomes reduces the subjectivity of analysing karyotypes using banding patterns when assessing karyotypic mutations. Compared to comparative genomic hybridisation (CGH), a method for analysing copy number variations relative to ploidy, MFISH has the ability to visualise large structural variants and balanced translocations. MFISH provides a robust method to understand the mutational landscape at a populational level. MFISH has largely been applied in the clinic to characterise human chromosome biology, such as numerical and structural variations within cancer patient samples. Other specific uses include understanding spontaneous micronucleation compared to irradiation induced mutations and identification of mutually exclusive gene amplifications in gastric cancer patients.

In one embodiment, the step of karyotyping the cells in a cell culture is carried out during metaphase. In a further embodiment, karyotyping comprises using multi-colour fluorescence in situ hybridisation (MFISH), Giesma banding (G banding), comparative genomic hybridisation (CGH) or spectral karyotyping (SKY). In one embodiment, karyotyping comprises using MFISH or G banding. In one embodiment, karyotyping is by MFISH. In one embodiment, the step of karyotyping the cells in a cell culture comprises performing quantitative fluorescence in situ hybridisation (Q-FISH). Q-FISH using peptide-nucleic acid probe may be used to analyse telomeres.

A genomic instability value is derived by applying a genomic instability metric to the karyotypes of the cells in a clonal population. A genomic instability metric is a metric by which the level of chromosomal aberrations within the genome of a cellular lineage may be assessed. The level of chromosomal aberrations within the genome of a cellular lineage may be assessed in different ways. Provided herein are two genomic instability metrics: (i) a percentage clonal chromosomal aberration (CCA) and/or percentage non-clonal chromosomal aberration for each clonal cell line (i.e. clonal population), and (ii) a standard deviation or variance of an average matching cost distribution of a clonal population.

Therefore, in one embodiment, the genomic instability value is obtained by deriving a percentage clonal chromosomal aberration (% CCA) and/or percentage non-clonal chromosomal aberration (% NCCA) for each clonal cell line. As such, in this embodiment, CCA and NCCA are the genomic instability metric used to derive the genomic instability value. CCA and NCCA is a general mutation metric that describes the overall mutation landscape within a cell line (Henry Heng et al, Molecular Cytogenetics, 2016).

A clonal chromosomal aberration is a chromosomal aberration which is detected at least twice within 20 to 40 randomly examined mitotic figures. In contrast, a non-clonal chromosomal aberration is a chromosomal aberration which is detected in only a single cell within 20 to 40 randomly examined mitotic figures. Therefore, taking 40 mitotic images, a CCA is a chromosomal aberration that occurs in 5% or more of the population, whereas an NCCA is a chromosomal aberration that occurs in less than 5% of the population.

The karyotypes of one or more cells grown from each clonal cell line may have the same chromosomal aberrations and, as such, the same karyotype. By grouping cells with the same or similar chromosomal characteristics (i.e. that have undergone same or similar mutational events), it is possible to group the cells in each cell culture, that is to say the population, into subpopulations by karyotype. In this way, using the number of cells (images) in a given subpopulation and based on the total population size (e.g. total number of images analysed for a given cell population) it is possible to assign each subpopulation as comprising CCA or NCCA.

Therefore, in one embodiment, the methods of the invention comprises a further step of determining subpopulations of the cells in each cell culture (i.e. cells grown from a clonal cell line) by karyotype. In another embodiment, the step of deriving the genomic instability value comprises assigning each subpopulation as comprising CCA or NCCA.

The inventors have identified a strong correlation between the overall % CCA and % NCCA in a clonal population and production stability and instability of the respective clonal cell line from which the clonal population is derived. A high percentage frequency of CCA populations correlates to productionally stable cell lines. Conversely, a greater percentage frequency of NCCA populations was retained in the unstable arm of the cell line panel. As such, the distinct groupings of % CCA and % NCCA for stable and unstable cell lines at an early time point indicate that this genomic metric can be utilised as a production stability predictor.

In one embodiment, a CCA is a chromosomal aberration which is detected in 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more of a clonal population. In one embodiment, CCA is a chromosomal aberration which is detected in 2% to 10% of a clonal population. In one embodiment, CCA is a chromosomal aberration which is detected in 5% to 10% of a clonal population. In one embodiment, CCA is a chromosomal aberration which is detected in 5% of a clonal population. In one embodiment, an NCCA is a chromosomal aberration which is detected in 5% or less, 4% or less, 3% or less, 2% or less or 1% or less of a clonal population. The skilled person would understand that the frequency of the CCA or NCCA in a clonal population as defined by the respective % CCA or NCCA will depend on the sample size of mitotic images examined.

In one embodiment, deriving the genomic instability value further comprises determining a percentage CCA and/or percentage NCCA in a population of cells for each clonal cell line.

The inventors have also identified that there is a distinct separation between productionally stable and unstable cell lines based on the variance or standard deviation of average matching cost distribution. As such, the variance or standard deviation of average matching cost is the genomic instability metric used to derive the genomic instability value. Therefore, in one embodiment, the genomic instability value is obtained by deriving a standard deviation of an average matching cost distribution. In another embodiment, the genomic instability value is obtained by deriving the variance of an average matching cost distribution.

A variance or standard deviation of the average matching cost distribution is used to quantify the amount of variation between sets of chromosomes based on the colour (i.e. fluorescent intensities) of the individual chromosomes, for example, as emitted by fluorescent probes. Based on the colour of the chromosomes, this metric allows for quantification of the frequency of different colour patterns across karyotypes in a clonal population.

A matching cost is the percentage discordance between the colours of a pair of chromosomes between two karyotypes (i.e. 2 images). A small matching cost represents similarity in the colour profile (genomic similarity) and a large matching cost represents genetic dissimilarity. A total matching cost for two karyotypes is the sum of the matching costs for the set of most colour-similar chromosome pairs, one from each karyotype.

To account for variation in numbers of chromosomes between cells in a cell line, the total matching cost for two cells is averaged over the number of chromosome pairs. An average matching cost for a pair of images (i.e. 2 karyotypes) is calculated by averaging the sum of the matching costs of all the pairs of corresponding chromosomes in said pair of images. Each karyotype (i.e. image) is compared against all the other karyotypes in a sample taken from the clonal population and for each pair of images compared, an average matching cost is obtained. In this way, a distribution of average matching costs is obtained for each clonal population. From this distribution, a variance or standard deviation is calculated to obtain the variance or standard deviation of the average matching cost distribution. The smaller the variance or standard deviation of the average matching cost distribution of a clonal cell line, the more genomically stable the respective clonal cell line is. The inventors have shown that the variance of the average matching cost distribution correlates well with % CCA/% NCCA.

Therefore, in one embodiment, the step of deriving a genomic instability value comprises determining an average matching cost distribution. In a further embodiment, the step of deriving a genomic instability value comprises determining a standard deviation of the average matching cost distribution. In another embodiment, the step of deriving a genomic instability value comprises determining a variance of the average matching cost distribution.

In some embodiments, matching costs may be used to determine subpopulations of a clonal cell line (i.e. subpopulations of a clonal population). As described above, a matching cost may be generated for each chromosome pair between two images. A low matching cost represents chromosomes that are similar based on their fluorescent colour make up within the chromosome mask. A mask is an overlay over an image to identify chromosomes and discount non-chromosome areas of the image. A high matching cost indicates a mutation event which has occurred as the fluorescent colour of one chromosome deviates significantly from the other chromosome. Therefore, a high matching cost identifies genetic mutations between two subpopulations. Each subsequent image can be assigned to either a new subpopulation or one that has already been identified, providing a frequency score of each population.

In some embodiments, the frequency of identified subpopulations can be calculated and designated into a clonal chromosomal aberration (genetically stable, CCA) and non-clonal (genetically unstable, NCCA) population. Alternatively, as outlined above, the variance or standard deviation of matching costs between cells in a cell line can also be used as a genomic stability metric, where an increased spread of the matching costs indicates a higher amount of chromosomal aberrations in the images analysed.

Once derived, the genomic instability values are used to identify productionally stable and/or productionally unstable clonal cell lines. Such identifications (i.e. predictions) are beneficial to cell line development timelines as it provides the means for triaging unstable cell lines at a much earlier time point (e.g. 10, 15 or 20 generations) as compared to determining the production stability of a clonal cell line after completing the whole stability assessment (70-150+/−10 generations).

The genomic instability values may be employed in different ways to either select for productionally stable cell lines or to filter out productionally unstable cell lines. One way is by ranking the % CCA, or variance or standard deviation (SD) of the average matching cost distribution. For example, top 6 and bottom 6 predictions based on the ranking of % CCA for each cell line has the potential to quickly identify stable (for cell line progression) and unstable (for triaging) clonal cell lines.

Alternatively, quartile predictions based on the genomic instability values may be used to readily identify the top 25% stable cell lines and the bottom 50% productionally unstable lines based on % CCA, or variance or SD of the average matching cost distribution. Robustly triaging the bottom 50% would drastically increase cell line development capacity by freeing up limited mini bio-reactor space.

Another prediction may be based on a % CCA threshold, or variance or SD of the average matching cost distribution threshold derived from the genomic instability values of clonal cell lines with known production stability/instability designation as a reference. The threshold may be a genomic instability value that separates productionally stable and productionally unstable clonal cell lines. A potential benefit of this prediction is that the threshold may be refined as more data is generated, providing a potential increased prediction accuracy rate.

In one embodiment, the % CCA threshold is ≥60%, ≥6.5%, ≥70%, ≥75%, ≥80%, ≥85%, ≥90%, ≥95%. In one embodiment, the % CCA threshold is 70%. That is to say, clonal cell lines with percentage CCA equal to or above 70% are productionally stable, whilst clonal cell lines with less than 70% CCA may be considered as unstable. In one embodiment, the % CCA threshold is 78%. In one embodiment, the % CCA threshold is between 60% to 95%. In one embodiment, the % CCA threshold is between 70% to 95%. In one embodiment, the % CCA threshold is between 75% to 95%. In one embodiment, the % CCA threshold is between 80% to 95%. In one embodiment, the % CCA threshold is between 85% to 95%. In one embodiment, the % CCA threshold is between 90% to 95%. In one embodiment, the % CCA threshold is 70%, 75%, 78%, 80%, 85% or 90%.

In one embodiment, the variance of the average matching cost distribution threshold is ≤100, ≤90, ≤80, ≤75, ≤70, ≤65, ≤60, ≤55, ≤50, ≤45, ≤40, ≤35, ≤30, ≤25, ≤20, ≤15, ≤10 or ≤5. That is to say that a variance equal or less to the identified variance threshold is considered to be productionally stable. In one embodiment, the variance of the average matching cost distribution threshold is ≤70. In one embodiment, the variance of the average matching cost distribution threshold is ≤65. In one embodiment, the variance of the average matching cost distribution threshold is ≤60. In one embodiment, the variance of the average matching cost distribution threshold is ≤55. In one embodiment, the variance of the average matching cost distribution threshold is ≤50. In one embodiment, the variance of the average matching cost distribution threshold is ≤45. In one embodiment, the variance of the average matching cost distribution threshold is ≤40. In one embodiment, the variance of the average matching cost distribution threshold is ≤35. In one embodiment, the variance of the average matching cost distribution threshold is ≤30. In one embodiment, the variance of the average matching cost distribution threshold is between 25 and 70. In one embodiment, the variance of the average matching cost distribution threshold is between 25 and 60. In one embodiment, the variance of the average matching cost distribution threshold is between 30 and 45.

In one embodiment, the SD of the average matching cost distribution threshold is ≤10, ≤9, ≤8, ≤7, ≤6.5, ≤6, ≤5.5, ≤5, ≤4.5, ≤4 or ≤3.5. That is to say that a variance equal or less to the identified SD threshold is considered to be productionally stable. In one embodiment, the SD of the average matching cost distribution threshold is ≤8. In one embodiment, the SD of the average matching cost distribution threshold is ≤7.5. In one embodiment, the SD of the average matching cost distribution threshold is ≤7. In one embodiment, the SD of the average matching cost distribution threshold is ≤6.5. In one embodiment, the SD of the average matching cost distribution threshold is ≤6. In one embodiment, the SD of the average matching cost distribution threshold is ≤5.5. In one embodiment, the SD of the average matching cost distribution threshold is ≤5. In one embodiment, the SD of the average matching cost distribution threshold is ≤4.5. In one embodiment, the SD of the average matching cost distribution threshold is ≤4. In one embodiment, the SD of the average matching cost distribution threshold is between 5 and 8.5. In one embodiment, the SD of the average matching cost distribution threshold is between 5 and 8. In one embodiment, the SD of the average matching cost distribution threshold is between 5.5 and 7.

In one embodiment, variance or SD of the average matching cost distribution threshold is calculated by building a decision tree on clonal cell line SD or variance of average matching cost distribution that are known to be productionally stable or unstable that best separates the two stability classes. The threshold identified by the decision tree can then be applied to variance or SD of average matching cost distribution of new cell lines. If the experimental protocol is modified, the threshold should be reviewed and re-estimated on new cell line MFISH images with known production stability outcomes if the threshold is deemed no longer fit for purpose.

In one embodiment of the invention, the step of predicting production stability and/or instability of the clonal cells in each cell culture comprises one or more of the following: i) ranking the clonal cells by % CCA, variance of the average matching cost distribution or SD of the average matching cost distribution; (ii) applying a % CCA threshold, variance of the average matching cost distribution threshold or SD of the average matching cost distribution threshold; and (iii) applying a quartile threshold. In one embodiment, the production stability and/or production instability in each cell culture is predicted by applying a % CCA threshold, or variance or SD of the average matching cost distribution threshold. In one embodiment, the % CCA threshold is ≥70%, ≥75%, ≥80%, ≥85%, ≥90%, ≥95%. In one embodiment, the % CCA threshold is 70%. In a further embodiment, the % CCA threshold is 78%. In one embodiment, the % CCA threshold is between 70% to 95%. In one embodiment, the % CCA threshold is 70%, 75%, 78%, 80%, 85% or 90%.

In one embodiment, the correct prediction rate is between about 60% to about 100%, about 70% to about 100%, about 80% to about 100%, or about 90% to about 100%. In one embodiment, the correct prediction rate is between about 70% to about 100%. In one embodiment, the correct prediction rate is about 60%, about 70%, about 80%, about 90% or about 100%.

In one embodiment, the correct prediction rate by ranking the clonal cells by % CCA, variance of the average matching cost distribution or SD of the average matching cost distribution is 83%. In one embodiment, the prediction rate of correctly identifying a productionally unstable cell line by ranking the clonal cells by % CCA, variance of the average matching cost distribution or SD of the average matching cost distribution is 100%. In one embodiment, the prediction rate of correctly identifying a productionally stable cell line by ranking the clonal cells by % CCA, variance of the average matching cost distribution or SD of the average matching cost distribution is 65%.

In one embodiment, the correct prediction rate by applying a % CCA threshold or variance of the average matching cost distribution or SD of the average matching cost distribution threshold is 80%. In one embodiment, the prediction rate of correctly identifying a productionally unstable cell line by applying a % CCA threshold, variance of the average matching cost distribution or SD of the average matching cost distribution threshold is 83%. In one embodiment, the prediction rate of correctly identifying a productionally stable cell line by applying a % CCA threshold, variance of the average matching cost distribution or SD of the average matching cost distribution threshold is 75%.

In one embodiment, the correct prediction rate by applying a % CCA quartile threshold variance of the average matching cost distribution quartile threshold or SD of the average matching cost distribution quartile threshold is 70%. In one embodiment, the prediction rate of correctly identifying a productionally unstable cell line by applying a % CCA quartile threshold (bottom 25%), variance of the average matching cost distribution quartile threshold (bottom 25%) or SD of the average matching cost distribution quartile threshold (bottom 25%) is 100%. In one embodiment, the prediction rate of correctly identifying a productionally stable cell line by applying % CCA quartile threshold (top 25%), variance of the average matching cost distribution quartile threshold (top 25%) or SD of the average matching cost distribution quartile threshold (top 25%) is 68%.

Various steps of the methods of the invention may be automated. In one embodiment, the step of karyotyping the cells in each cell culture and/or the step of deriving a genomic instability value from the karyotyping is/are automated. In one embodiment, the step of karyotyping of the cells may be automated.

In one embodiment, the automation is computer-implemented automation. The automation is typically achieved through computer-implementation (i.e. it is a computer-implemented step). The computer-implementation may involve an image classification system. The computer-implemented step or the image classification system may comprise a machine learning system, for example an artificial neural network, more particularly a convolutional neural network.

Automated processes may remove any subjectivity associated with manual image analysis for during karyotyping and/or deriving a genomic instability value. Therefore, in one embodiment, the step of karyotyping the cells in each cell culture and/or the step of deriving a genomic instability value from the karyotyping comprises automated image analysis.

In one embodiment, image analysis may be automated by using a software. Image analysis is often performed using software that allows characterisation of fluorescent images. An example is CellProfiler™. Stained (e.g. fluorescent) images may be analysed using a CellProfiler™ workflow to extract fluorescent intensities from individual chromosomes so as to be able to correlate fluorescent pixel intensities to individual chromosomes within an image. The images may undergo threshold corrections to remove background fluorescence.

In one embodiment, automation of image analysis comprises segmentation of chromosomes in an image. Faithfully segmenting chromosomes within images is a critical step in an automation pipeline. Presence of artefacts, differences in illumination and proximal chromosomes within an image present a number of challenges to the segmentation. To overcome these challenges, a deep learning-based approach (DL) to derive the masks may be included in the automation process. A mask is an overlay over an image to identify chromosomes and discount non-chromosome areas of the image. Segmented chromosome pixels can be coloured according to the fluorescent signals.

Colouring of the chromosomes may be by a pre-trained Gaussian mixture model applied to the fluorescent intensities, which further classifies the fluorescent intensities into one of a set of pre-determined pseudo-colour classes.

In an automated process for image analysis, a chromosome may be characterised by a multicolored pie chart, wherein the size of a pseudo-coloured sector reflects the proportion of the chromosome's pixels classified as that colour. Given two segmented and pseudo-coloured images, a set of chromosome-to-chromosome pairs, one from each image, can be derived by calculating a cost matrix whose rows and columns are indexed by the chromosomes of image 1 and image 2 respectively, and whose ij-^{th} entry is the cost of matching chromosome i from image 1 to chromosome j from image 2; and solving the linear assignment problem for this cost matrix. The solution to the linear assignment problem is a set of chromosome-to-chromosome pairs that yield the lowest total matching cost. The average of this total matching cost, taken over the number of successfully paired chromosomes, provides an indication of whether the two chromosomal populations have the same or similar karyotype.

In one embodiment, each cell line is assessed for genomic stability by computing the average matching cost for each pair of images, forming an average matching cost distribution and calculating the variance or standard deviation (SD) of this distribution. This variance or standard deviation correlates with the % CCA metric.

In another embodiment, each cell line is assessed for genomic stability by computing the average matching cost for each pair of images, forming an average matching cost distribution and calculating the variance of this distribution.

The genomic instability of a clonal cell line may be assessed by analysing the telomeres of the chromosomes. In one embodiment, karyotyping comprises analysing the telomeres of the chromosomes. In one embodiment, analysing the telomeres of the chromosomes comprises quantitative fluorescence in situ hybridisation (Q-FISH).

In normal homeostasis, telomeres are situated at the extreme ends of chromosomes.

Telomeres are formed by G-rich repeats (TTAGGGn) and protected by shelterin, a 6-membered protein complex, which binds specifically to telomeres and inhibits DNA damage pathways at the single stranded telomeric DNA through its sequestering by POT1 (de Lange, 2005). In Chinese Hamster Ovary (CHO) cell lines, interstitial telomere sequences (ITS) are in abundance compared to the extremities. Shelterin complex is known to bind to ITS, however, its inhibitory action on localised DNA damage is less well defined (Schmutz and de Lange, 2016).

At the extreme ends, upon cellular division, telomere length shortens until they reach the Hayflick limit, the critical length of telomeres where apoptosis is triggered (Hayflick, 1965; Hayflick and Moorhead, 1961). Shortening of the telomeres to this critical length results in a significant loss of shelterin complex and de-protection of the ssDNA leading to the activation of DNA damage response (DDR) pathways. DDR pathways, through the action of Ataxia-telangiectasia mutated (ATM) and ataxia telangiectasia and Rad3-related protein (ATR), usually lead to genetic insult repair before progression through mitosis by inhibition of CDK proteins that slow down cell cycle progression (Huen and Chen, 2008). Upon repair, cell cycle progresses without the activation of apoptotic pathways (Roos and Kaina, 2006).

CHO cells represent a highly proliferative and immortalised cell line, reminiscent of cancer cell lines such as HeLa and indirectly HEK293T. Although not derived from a cancer tissue, HEK293T cells express Ad5 E1A/E1B proteins which deregulate pRetinoblastoma (RB) and p53 pathways, disrupting the cell cycle (Berk, 2005; Sha et al., 2010). If genetic insults are not corrected appropriately and the immortalised cell line has acquired mutations that allow cell cycle progression, genetic instability can occur. If a genetic insult occurs specifically at the telomeres, tumour suppressor p53 binding protein (TP53BP1) is recruited and facilitates non-homologous end joining (NHEJ) at chromosomal ends. TP53BP1 action is only made possible in absence of p53 and RB pathways (O'Sullivan and Karlseder, 2010).

Cells that obtain fused chromosomes and acquired the ability to pass through mitosis lead to break-fusion-bridge (BFB) cycles whereby chromosomes break non-reciprocally to create two genetically distinct daughters (Marotta et al., 2013). BFB cycles have been implicated to intratumor heterogeneity and been shown to promote DNA amplification and chromosome loss (Gisselsson et al., 2000; Lo et al., 2002; Thomas et al., 2018). This may represent a pathway that leads to the genomic instability of CHO cell lines (Vcelar et al., 2018a; Vcelar et al., 2018b).

Mammalian cells such as CHO (Chinese Hamster Ovarian), BHK, NS0, Jurkat, K562, HeLa or PerC6 are routinely employed within the biopharmaceutical industry to manufacture biopharmaceuticals. These cells are genetically engineered and then selected in such a way as to ensure that high titre expression of the desired protein is observed when the resulting cell lines are cultured in bioreactors. Such host cells may also contain advantageous genotypic and/or phenotypic modifications e.g. the CHO-DG44 host strain has copies of its dhfr gene disabled, whilst other hosts might have the glutamine synthetase genes disabled (e.g. CHOK1a-GS-KO). Alternative modifications may be to the enzyme machinery involved in protein glycosylation. Others may have advantageous genotypic and/or phenotypic modifications to host apoptosis, expression and survival pathways. These and other modifications of the host alone or in combination, can be generated by standard techniques such as over-expression of non-host or host genes, gene knock-out approaches, gene silencing approaches (e.g. siRNA), or evolution and selection of sub-strains with desired phenotypes. Such techniques are well established in the art.

In one embodiment, the clonal cell line is a mammalian cell line. In one embodiment, the mammalian cells is a CHO (Chinese Hamster Ovarian) cell, BHK cell, NS0 cell, Jurkat cell, K562 cell, HeLa cell or PerC6 cell. In one embodiment, the mammalian cell is a CHO cell. In one embodiment, the mammalian cell is a CHOK1 cell. In one embodiment, the CHO cell line is a glutamine synthetase (GS) is knock out cell. In one embodiment, the mammalian cell is CHOK1a-GS-KO.

The invention will now be described in further detail with reference to the following, non-limiting Examples.

### EXAMPLES

### Example 1: Methods

### Cell Culture

### Cell Lines

Cell lines for which production stability had already been determined were obtained from liquid nitrogen stocks from GlaxoSmithKline (GSK). Cell lines used were CHOK1a-GS-KO, CHOK1a, DG44, HEK293T and CHOK1a-GS-KO (Protein 2, 3, 4 and 5). Cell lines for therapeutic proteins 2, 3 and 5 were used for productionally stable and unstable comparison experiments and protein 4 was used for blinded validation of MFISH production stability prediction method.

### Viable Cell Counting

500 μl of cell suspension were decanted into a 4 ml sampling tube. 500 μl if TrypLE (Gibco, #12605010) is added to the cell suspension and sample processed by a Vi-Cell XR (BeckmanCoulter), providing metrics of total and viable cell counts, percent viability and cell diameter.

### Cell Line Thawing

Cell vials were thawed in 37° C. PBS and resuspended in 10 mL of media. Cell lines were counted on a ViCell (Beckman Coulter) by adding 500 μL TrypLE (Gibco, #12605036) to 500 μL cell suspension. Culture flasks were seeded with 0.5×10\hat{ }6 cells in 20 mL of media and incubated in a humidified shaking incubator set at 37° C., 5% CO₂ and 140 rpm.

### Cell Culture Maintenance

Upon recovery of cells to >95%, cell lines were maintained and passaged in medium supplemented with nutrients+25 μM MSX at 0.3×10⁶ cells in 30 mL every 3 or 4 days. Seeding density was calculated using a ViCell (Beckman Coulter).

### Cytogenetics

### Chromosome Harvest

0.5 mL of cells per cell line was added to T25 flasks containing 5 mL of fresh media. Cells were placed into a static incubator (37° C., 5% CO₂) and cultured for three days. 2 mL of media was replaced by 2 mL of fresh media in each T25 and 100 μl of KaryoMAX colcemid (Gibco, #15212012) was added and T25s were placed into the shaking incubator (37° C., 5% CO₂) overnight.

Cells were then spun down at 1200 rpm for 5 minutes at room temperature (RT), supernatant discarded, and the pellet resuspended with 5 mL of warm (37° C.) 0.075M KCL (Sigma, #P5405) and placed into a static incubator (37° C.) for 5 minutes. 2 mL of pre-chilled fixing solution (−20° C.), 3:1 solution of methanol (Sigma, #34860): acetic acid (Sigma, #A6283) was added and the cells spun down at 1200 rpm for 5 minutes.

The supernatant was discarded, and the pellet resuspended in 5 mL fixing solution, then incubated at −20° C. for 30 minutes. Cells were spun down and resuspended in an appropriate volume/density to apply the metaphase spreads to slides. Slides were then stored at −20° C. until probes were applied.

### Telomere Fluorescent In-Situ Hybridisation

Slides containing sample metaphases were placed into a coplin jar containing 40 ml TBS solution (Agilent Dako, K532711-8) and incubated at room temperature for 2 minutes. Slides were placed into another coplin jar containing 40 ml TBS solution and incubated for a further 2 minutes. Slides were treated with an ethanol series of 70%, 90% and 100% for two minutes each. Slides were removed from the chambers and left to dry.

5 μl of Telomere probe (Agilent Dako, K532711-8) was added to the slides and covered with 18×18 mm cover glass and sealed with fixogum (VWR, ICNA11FIXO0125). Slides were placed upright into a humidified chamber (ThermoBrite) and incubated for two hours at 37° C. Slides were removed from the humidified chamber and fixogum and cover slip removed. Slides were placed into a coplin jar containing 40 ml rinse solution (Agilent Dako, K532711-8) and incubated for 2 minutes.

Slides were then incubated in 40 ml wash solution (Agilent Dako, K532711-8) for 5 minutes at 65° C. Slides were treated by an ethanol series of 70%, 90% and 100% for 2 minutes each. Slides were allowed to dry and prewarmed (37° C.) 20 μl DAPI II counterstain (Abbott Molecular, 06J50-001) applied. Slides were covered with a 22×50 mm cover slide and sealed with fixogum. Images were captured using an Axio Z2 imager using metasystems software (V5.7.4).

### Telomere FISH Performed on Thermo Brite Elite (Leica Biosystems)

Slides containing sample metaphases were placed face down into the incubation chamber. 30 ml TBS solution was added per chamber and incubated at room temperature for 2 minutes under rocking conditions (12/min). Chambers were drained and TBS is re-added to the chambers and incubated for a further 2 minutes under rocking conditions.

Slides were treated with an ethanol series of 70%, 90% and 100% for two minutes each. Slides were removed from the chambers and left to dry. 5 μl of Telomere probe (Agilent Dako, K532711-8) was added to the slides and covered with 18×18 mm cover glass and sealed with fixogum (VWR, ICNA11FIXO0125). Slides were placed upright into the chambers and chambers were filled with water and incubated for two hours at 37° C. Slides were removed from the chambers and fixogum and cover slip removed.

Slides were placed face down into the chamber and chambers were filled with 30 ml rinse solution and incubated for 2 minutes. Chambers were drained and filled with 30 ml per chamber of wash solution and slides were incubated for 5 minutes at 65° C. Chambers were drained and slides were treated by an ethanol series of 70%, 90% and 100% for 2 minutes each. Slides were left to dry and prewarmed (37° C.) 20 μl DAPI II counterstain (Abbott Molecular, 06J50-001) applied. Slides were covered with a 22×50 mm cover slide and sealed with rubber cement. Images were captured using an Axio Z2 imager using metasystems software (V5.7.4).

### Multicolour-FISH (MFISH)

MFISH was performed using Metasystems 12×CHamster (D-1526-060-DI) probe set. In brief, coplin jars with 0.1×SSC (Invitrogen, #15557044) and 2×SSC were placed at 4° C., with an additional 2×SSC prewarmed at 70° C. Slides were placed into 70° C. 2×SSC for 30 minutes, then removed from the water bath and left to cool for 20 minutes. During this step, 5 μl per slide of 12×CHamster probes was prepared in a PCR machine using a program of 75° C. for 5 minutes, 10° C. for 30 seconds, 37° C. for 30 minutes.

Slides were then transferred to 0.1×SSC at room temperature (RT) for 1 minutes and denatured in 0.07N NaOH (Sigma, #S2770) at RT for 1 minute subsequently. Slides were then placed sequentially into 0.1×SSC and 2×SSC at 4° C. for 1 minute each and dehydrated in an ethanol (Sigma, #51976) series of 70%, 80%, 90% and 100% for 1 minute each. After air drying, 5 μl of denatured and prehybridized probe was placed onto metaphase spreads, overlaid with a coverslip and sealed with rubber cement. Slides were incubated in a humidified chamber (ThermoBrite, Leica Biosystems) at 37° C. for 1-2 days.

After incubation the rubber cement and coverslips were removed, and slides were placed into prewarmed (72° C.) 0.4×SSC for 2 minutes. Slides were then placed in 2×SSCT (2×SSC, pH 7-7.5 containing 0.05% Tween20) at RT for 1-2 minutes. Slides were washed briefly in double distilled water to avoid crystal formation and air dried. 20 μl of DAPI/antifade (D-0902-500-DA) was applied to metaphases and a coverslip overlaid. Images were captured using Metasystems automated acquisition platform. The software was programmed to capture 6 individual colour channels (DAPI, aqua, green, orange, red and gold) and images analysed as outlined in the population determining section.

### Multicolour-FISH (MFISH) Performed on Thermobrite Elite (Leica Biosystems)

MFISH was performed using Metasystems 12×CHamster (D-1526-060-DI) probe set and Thermo Brite Elite. Slides containing sample metaphases were placed face down into the incubation chamber. 30 ml of 2×SCC+0.05% Tween20 solution is added per chamber and incubated at 37° C. for 30 minutes under rocking conditions (12/min). 5 μl per slide of 12×CHamster probes was prepared in a PCR machine using a program of 75° C. for 5 minutes, 10° C. for 30 seconds, 37° C. for 30 minutes.

Chambers were drained and demi-water was added to the chambers and incubated for 30 seconds, under rocking conditions. Demi-water wash was repeated a second time. 30 ml 0.07N NaOH was then added to chambers and incubated for 1 minute, under rocking conditions. Chambers were drained, then ice cold 0.1×SCC was added to chambers and incubated for 1 minute. Subsequently ice cold 2×SCC was added and incubated for 1 minute. Slides were washed with demi-water for 30 seconds. Slides then enter an ethanol series comprising of 70%, 95% and 100% ethanol.

Slides were removed from the chambers and left to dry until ethanol has evaporated. Probes prepared earlier were then applied to the metaphases and covered with a coverslip and rubber cement. Slides were then hybridised within the chambers, upright and overnight, in 30 ml demi-water at 37° C. Cover slips were removed and placed face down into the chambers. 30 ml 0.4×SSC was added to the chambers and incubated for 2 minutes at 68° C. Chambers were drained and then re-filled with 30 ml of 2×SSC and 0.05% Tween20 solution and incubated for 2 minutes at 25° C. Chambers were drained and slides were then treated in an ethanol series containing 70%, 80% and 100% ethanol. Slides were removed and excess ethanol was allowed to evaporate. 20 μl antifade DAPI (Metasystems D-0902-500-DA) was added to slides and a coverslip placed on top, sealed with rubber cement. Slides were then imaged using Axio Imager Z.2.

### Image Analysis

### Population Determining

Subpopulations were elucidated through analysing each individual image, which represents a single cell.

A new subpopulation was defined by witnessing a mutagenic event (such as a translocation). In this example, a translocation was confirmed by reviewing the DAPI channel image to ensure that the chromosomes were attached to each other and not just in close proximity. Additionally, the colour percentage change between the mean chromosome colour profile (chromosome percentage colour across all non-mutated chromosomes) and the mutated chromosome colour profile was confirmed using the mean fluorescent intensities extracted by the CellProfiler™ (https://cellprofiler.org/).

Missing or increased numbers of chromosomes must be confirmed by witnessing 3 metaphase spreads that contain the same aberration. This is to ensure that the aberration is not an artefact of the metaphase spread preparation—as outlined in the European Cytogenetics association guidelines (https://www.e-c-a.eu/en/GUIDELINES.html).

The frequency of each metaphase belonging to each population was recorded to determine whether the aberration is clonal or non-clonal. A clonal chromosomal aberration (CCA) is defined as a subpopulation that comprises >5% of the total population and considered as a chromosomally stable subpopulation, as it has established itself as a dominant population. Non-clonal chromosomal aberrations (NCCA) were defined as <=5% of the total population (Henry Heng et al, Molecular Cytogenetics, 2016). Increased numbers of NCCA5 in the total population may indicate an increasing mutagenic background leading to chromosomal instability.

Confirmation of mutations using Cell Profiler

The following workflow is performed on CellProfiler™. Single channel images covering DAPI, green, red, gold, orange, and aqua filter sets were exported from the Metafer software (Metasystems, V5.7.4) in .tif format. Images were selected based on their spread as chromosomes that were in close proximity or cross each other do not segment well with this workflow. 6 single channel images were thresholded using the thresholding module with the Global-Otsu algorithm selected using a 1.1 correction factor threshold value. Images were smoothened after thresholding, using a Gaussian filter.

Edges of the image were enhanced to improve identification of chromosomes by using a Sobel algorithm module. Identify primary objects is used to identify chromosomes within an image using the modules automatic threshold strategy. Resulting image masks were then manually edited using the edit image module with DAPI channel image as a guide, to allow for faithful masking of the original image. Each chromosome was arbitrarily assigned a number and this chromosome identifier stayed consistent throughout all populations analysed. Even if a mutagenic event has occurred the chromosome number will remain consistent. Fluorescent intensity values of each chromosome and single channel were extracted using the measure object intensity module. Fluorescent intensities were converted into percentages with the sum of all channels equalling 100%. Mutations within chromosomes that have visually been identified were then confirmed using the fluorescent intensities colour combinations extracted by this method.

### Telomere Length Quantification Using CellProfiler™

The following workflow was performed on CellProfiler™. Single channel images were thresholded using the thresholding module with the global Otsu algorithm selected using a 1.1 threshold correction value. Identify primary objects was used to identify chromosomes within an image using the automated strategy within the module. Image masks were manually edited using the edit mask module to ensure faithful masking of the image. Telomere signals were then identified within the chromosomes with the identify secondary objects using the global Otsu algorithm. The two thresholded images were related using the relate images module, to ensure fluorescent intensity of telomeres is only calculated within the chromosome regions. Values of the number of chromosomes and fluorescent intensity of telomeres within said chromosomes were extracted by the measure object intensity module.

### Chromosome Number Counting

Chromosome number counting was performed using Fiji (image J, version 1.51) by utilising the cell counter module of the software. 50 images of each time point were loaded into Fiji and the cell counter initialised. Images containing appropriately spread metaphase chromosomes were used to ensure all chromosomes were derived from a single cell source. After counting, analysed images were saved to include the counter markers.

### Data Analysis and Graph Generation

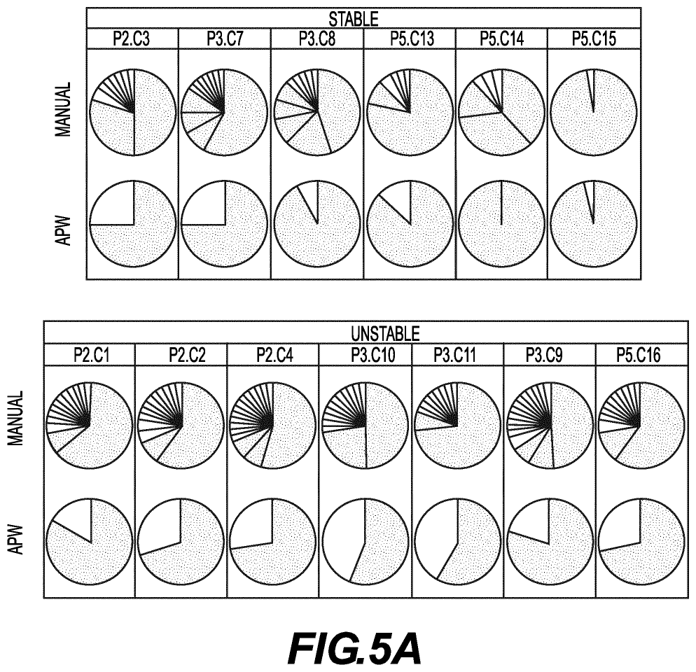

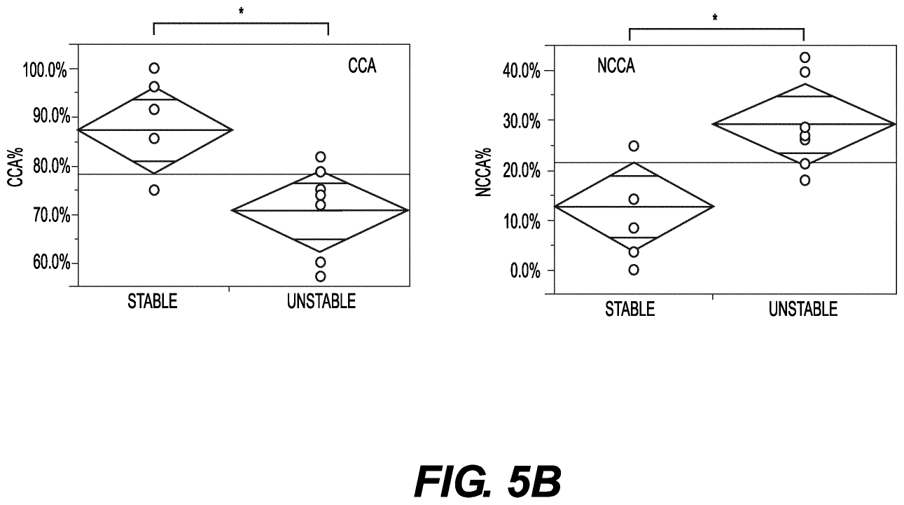

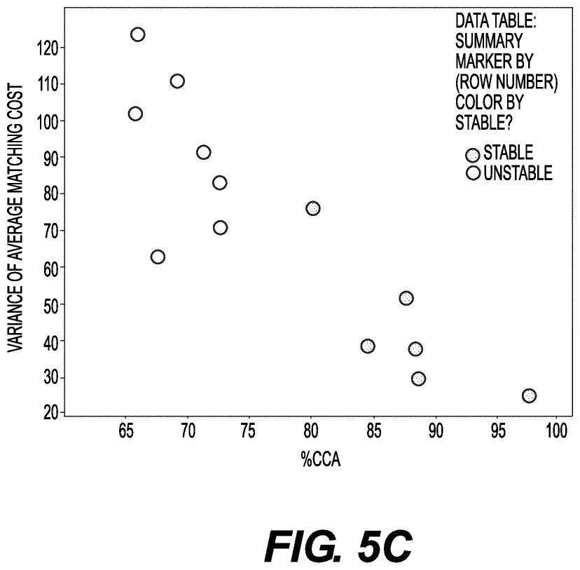

All graphs presented here were produced with JMP software (version 14), unless otherwise stated. The scatter plot of Variance of average matching cost distribution v % CCA (FIG. 5c) was produced using Tibco Spotfire.

### Statistical Analyses

All statistical analysis was performed with either JMP or InVivoStat software (version 3.7).

### Example 2: Baseline Characterisation of Host Cell Line (Telomeric and Mutational Baseline Profiles)

To elucidate a potential pathway, based on telomeric promoted genetic instability that may drive the CHO therapeutic protein production instability phenotype, a characterisation of the host cell line was performed. CHOK1 host variants were assessed by their telomeric profiles and compared to a cancerous-like cell line—HEK293T. The baseline of these results was used to compare telomeric profiles of the host cell line (without gene of interest) to therapeutic protein expressing cell lines, to assess any changes that may occur during the cell line development process of a therapeutic protein producing cell line. As CHOK1a-GS-KO host was used for subsequent analyses of productionally stable vs unstable cell lines, CHOK1a-GS-KO host was further analysed for baseline chromosomal mutations and telomere protection profiles.

### Telomere FISH Profiles of CHOK1a, CHOK1a-GS-KO and HEK293T Cell Lines Across a 6-Month Stability Assessment

Telomere sequence profiles were qualitatively assessed and compared against HEK293T cell line. HEK293 represents a normal telomeric signal profile expected in mammalian cell lines and is therefore used as a reference point when analysing CHO host lines. Changes in telomeric profile, across the 6-month culture period, may represent an indication of genomic instability.

CHOK1a, CHOK1a-GS-KO, DG44, and HEK293T cell lines were thawed and revived in media. Once cells had reached >98% viability, chromosomes were harvested from each cell line at passage 6. Chromosome harvesting was performed in 10 passage increments to mimic six months of cell culture, as performed in therapeutic protein production stability assessments. This mock stability assessment using commonly used CHOK1 hosts was performed to elucidate whether there are significant changes in telomere profile across the culturing period.

Compared to HEK293T, all CHOK1 host variants have most telomere sequences interstitially, with varying degrees of distinct patterns between each CHOK1 host. CHOK1 has a large block of TTAGGGn repeats on one chromosome, compared to CHOK1-GS-KO which has a telomere pattern that indicates BFB cycles may have occurred leading to non-reciprocal translocations or amplifications. Noticeably upon thresholding, there are no visible telomere signals at the extreme ends of the chromosome, whist interstitial telomeric repeats exist in large blocks of repeats. Lack of telomere sequence at the extreme ends of chromosomes may lead to increased telomere specific DNA damage response pathway activation promoting CHO chromosomal instability. Further analyses based on therapeutic producing proteins are derived from CHOK1-GS-KO. The cell line was characterised further to establish a baseline comparison against the therapeutic protein producing cell lines.

### Chromosome Number Distribution and Telomere FISH Quantification of CHOK1a-GS-KO Host Cell Line Across a 6-Month Stability Period

Chromosome number distribution and telomere sequence fluorescent signals were quantified across a 6-month stability period to generate a baseline characterisation of CHOK1a-GS-KO host cell line to be utilised as a comparator against CHOK1a-GS-KO therapeutic protein producing cell lines. Host cell lines should be telomerically stable to promote genomic stability during the manufacturing process. Fluctuations in chromosome number and telomere length may suggest an increase in genetic instability within the host over the 6-month stability period.

After thawing from a cell bank, CHOK1a-GS-KO host cell line was passaged until viability reached >98% and chromosomes were harvested and counted. At the early timepoint the median chromosome number was 19, which is reflective of previous reports (Vcelar et al., 2018a; Vcelar et al., 2018b). Modal chromosome range was between 18-21 chromosomes with an overall chromosome number range of 15 to 37. Outlier frequency of the modal chromosome range occurred in 7 cells with most of the data distributed between the model range (43). Conversely, at the late timepoint the median chromosome number increased to 20 (2-sample T-test, P=0.0384), indicating a gain of an additional chromosome across the 6-month culturing period.

Modal chromosome range remained the same, however there was an increase in the overall chromosome number range (7-39 chromosomes), and outlier frequency (12). This may suggest increased chromosome instability across the 6-month stability period, as there is an increase in cells that obtain an abnormal number of chromosomes. If this can be attributed to chromosomal instability, this data suggests that it is innate to the host cell line.

Using CellProfiler™, a semi-automated telomere quantification workflow was created to analyse telomere fluorescent intensities. Telomere probes are formed of PNA-TTAGGG(n) repeats with a conjugated Cy3 fluorophore. Fluorescent intensity is proportional to telomere signal and changes in fluorescent intensity is in relation to telomere sequence changes present within the chromosomes. Telomere signals that reside within the chromosome masks generated on DAPI images was measured, providing quantifications of telomere length that is chromosome specific.

50 images were analysed per timepoint and ratios of telomere fluorescent intensity to DAPI intensity (telomere proportion %) were compared between time points. The relative signals can be used to assess whether there has been a change in telomere length across the stability period. Mean telomere proportion obtained at the early time point was 2.9%. Over 6 months of continuous culture, mean telomere proportion increased to 8.9% (T-test, P=<0.0001). Telomere signals quantified solely resided within the chromosomes as interstitial telomere repeats, suggesting that the amplification of these sequences remained within the chromosome (ITS amplification) itself rather than at the extremities. This was visually confirmed by inspecting the late images for telomere signals at the extreme end of chromosomes.

### Results

Data presented here suggests there is an inherent genetic instability within the CHOK1a-GS-KO host. This is corroborated by the increase in median chromosome number across 6 months of culture (2-sample T-test, P=0.0384) that suggests a shift towards a dominant chromosomal population that obtains an additional chromosome. Moreover, an increase in telomere sequence (P=<0.0001), suggesting amplifications have taken place at interstitial telomere repeats has occurred. These traits are indicative of genetic instability at the chromosomal level, as highlighted in previous studies (Gisselsson et al., 2000; Lo et al., 2002; Thomas et al., 2018).

### Using Multi-Colour Fluorescent In-Situ Hybridisation (MFISH) to Assess Karyotypic Changes of CHOK1a-GS-KO Host Across a 6-Month Stability Period

Homogeneity of CHOK1a-GS-KO karyotype and fluctuation of karyotype over time were assessed. Host cell lines used for therapeutic protein production should retain genetic homogeneity from single cell cloning and maintain genetic stability during routine culture. Heterogeneity found at the host level may be passed onto derived producing cell lines.

Multi-colour fluorescent in-situ hybridisation (MFISH) was performed on CHOK1a-GS-KO cell lines at early (around 20 generations) and late timepoints (around 150 generations). MFISH ‘paints’ chromosomes to allow the visualisation of chromosome constituents. Probes specific to the Chinese hamster genome were generated against a primary cell line (Metasystems) and provides a colour code for each individual chromosome (e.g. chromosome 1=red, 2=brown, etc). Thus, it provides the means to assess chromosomal mutations within host cell cultures and allows comparison both internally, between cell lines and time points. Mutations can be tracked at a single cell level and specific chromosome mutations may be able to be attributed to phenotypic traits. Cell culture populations were manually determined using the methodology previously described.

Karyotypically distinct cells obtain a unique subpopulation ID and matching karyotypes are grouped together under the same subpopulation identifier. 40 randomly selected images were analysed, and the frequency of each subpopulation assessed. Based on this frequency, clonal chromosomal aberration (CCA, >5%) or non-clonal chromosomal aberration (NCCA, <=5%) was assigned to each subpopulation, reflecting on the population's genetic stability.

At an early timepoint, 18 distinct subpopulations were identified with subpopulation 1 and 2 representing the majority of the culture with 45% and 13%, respectively. Subpopulation 1 and 2 were designated CCA populations, whereas 15 subpopulations had a frequency of <=5% and were classed as NCCA populations. Analysing karyotype subpopulations after 6 months of continuous culture revealed 16 distinct subpopulations, suggesting 2 subpopulations had been lost during the culture process, although this may be an artefact of the number of images analysed. 3/16 subpopulations were designated CCA subpopulations compared to 13 NCCA. 6 out of the original 18 subpopulations were maintained throughout the 6-month process, with 10 de novo subpopulations arising. Although NCCA subpopulations 6, 8, 13 and 14 were maintained over the culture period, their NCCA designation did not change, suggesting that their acquired mutation did not provide a growth advantage.

Of the 10 de novo subpopulations, subpopulation 4 became dominant in the host culture. De novo subpopulation 4 gained a proliferative advantage and became the second largest subpopulation within the culture, surpassing the retained subpopulation 2 from the early timepoint. Subpopulation 2 frequency decreased from 13% to 8%, whilst subpopulation 4 obtained a 15% frequency. Comparisons of karyotypes between early and late timepoints revealed early subpopulation 2 may have been the pre-requisite of de novo subpopulation 4 as their karyotypes are identical apart from an apparent duplication of chromosome 6.

Duplication of chromosome 6 may have provided a proliferative advantage for the cell to allow it to establish itself as a predominate population within the flask.

Chromosome mutations that led to the creation of a newly distinct subpopulation was quantified. Chromosomes 2, 4, 5, 7, 10, 11, 14, 15, 18, 19 did not obtain any translocations that created a new population over the 6-month culture period, suggesting the majority of CHOK1a-GS-KO host chromosomes had maintained genomic stability. Chromosome 8 was the most frequently mutated compared to any other chromosome, accounting for 11 distinct populations across both time points, suggesting an inherent instability within this chromosome that contributes to the natural heterogeneity of the CHOK1a-GS-KO host cell line. There was a noticeable mutation increase in chromosome 6 and 13 (in addition to chromosome 8) after 6 months of continuous culture that contributed to 7 newly distinct populations (13 populations in total, when including chromosome 8).

Inherent weaknesses within chromosome 6, 8 and 13 may provide a mechanism by which mutations can be elicited to gain competitive advantage. This is corroborated by the duplication of chromosome 6 that allowed de novo subpopulation 4 to establish itself as the second most prominent population at the end of the culturing period (FIG. 6a, b, c, d, e).

Chromosome mutations were categorised into mutation types and coloured by chromosome to assess the predominant mode of mutation that creates heterogeneity within the host. Translocations in chromosome 1, 8, 9, 12 and 16 at the early timepoint and chromosome 3, 6, 8 and 13 at the late timepoint contributed to 19 newly distinct populations. Deletions (chromosome 8 and 13) and chromosomal breaks (chromosome 3 and 6) only occurred during the late time point, suggesting that these mutations may be indicative of long-term culture stress.

When analysing the overall CCA and NCCA frequency of populations within each time point, the ratio of CCA to NCCA remained similar. CCA frequency increased from 57.5% to 67.5% from early to late time points, suggesting a small shift to genetic stability through the creation of de novo subpopulation 4 which contributed to the CCA increase. Conversely, NCCA decreased from 42.5% to 32.5%, due to the increase in subpopulation 4 and the loss of two NCCA subpopulations from the early timepoint.

Overall, the data presented here highlights a single cell cloned host that has acquired mutations during routine culturing at both early and late time points. Prolonged culturing of the host seems to exacerbate this issue, maintaining the genomic heterogeneity as shown at the early stages of culturing. Chromosome 8 seems to play a role in the creation and maintenance of karyotypic heterogeneity, with translocations being the predominate type of mutation that creates de novo populations. Transfecting therapeutic proteins into a heterogenous host creates a scenario where upon single cell sorting, the clonal outgrowths will be genetically dissimilar as the plasmid may enter any one of the distinct subpopulations. In this manner, the background genomic heterogeneity of the host cell creates an environment where clones, single cell sorted from the same host, may have divergent CHO′mic profiles that may impact phenotypes in manufacturing conditions.

### Example 3: Characterisation and Comparison of Productionally Stable and Unstable Cell Lines to Identify Differential Patterns that Identify Causalities for the Production Instability Phenotype

### Chromosome Number Distribution and Relative Telomere Length Changes Between Stable and Unstable Therapeutic Protein Producing Cell Lines, Across Early and Late Time Points.

Chromosome number distribution within CHOK1a-GS-KO host obtained a median chromosome number of 19 and 20 at early and late time points, respectively. At both time points, a large range of chromosome numbers was observed. This was investigated within CHOK1a-GS-KO producing cell lines to assess any fluctuations of chromosome number between productionally stable and unstable groups. Additionally, telomere length was shown to increase over time, therefore ITS length was quantified in stable and unstable producing cell lines, across early and late time points, to assess whether there are any ITS length changes between the different groups.

### Results

Following on from the CHOK1a-GS-KO host characterisation, 18 cell lines producing three therapeutic proteins (protein 2, 3 and 5) were characterised for their chromosome distribution and telomere length. Cell lines were selected based on their production stability that was previously elucidated using Ambr 15s—an industry standard mini-bioreactor used to assess production stability. Stability is defined here as being able to produce the same level of titre within a +/−30% max titre loss threshold, across a 6-month production window.

To understand whether there are fundamental chromosome number differences that may be indicative of a productionally stable or unstable cell line, chromosome numbers were quantified. 14 out of 18 cell lines retained a median chromosome number of 19 or 20 that reflects the CHOK1a-GS-KO host cell line. 4 out of 18 cell lines had a median chromosome number between 35 and 38 chromosomes, suggesting that the single cell sorted clone was derived from a transfected cell in the host cell line that obtained a ‘aneuploid’ number of chromosomes (Table 1). ‘Aneuploid’ cell lines had the greatest spread of chromosome number with 90% confidence interval (CI) ranges spreading between 17 to 41 chromosomes, indicating that these cell lines have largely heterogenous karyotypes compared to ‘haploid’ cell lines.

Interestingly, 3 of the 4 cell lines that are considered ‘diploid’ were productionally stable, suggesting the increased genetic material provides a mechanism that allows the cell lines to cope better with production stress across the stability assessment period. Both the modal and 90% CI range of chromosome number distribution were similar comparing back to the host cell line, suggesting the use of a selection agent does not have a drastic impact on chromosome numbers.

A 2-way ANOVA approach was utilised to compare the median numbers of cell lines within different therapeutic proteins, to assess any significant difference between stable and unstable groups. Comparing therapeutic protein and stability as factors, there was no significant difference (P=0.108). Pairwise comparisons using a planned comparison approach (Table 2), where pairwise comparisons are first unadjusted and then a post-hoc test is applied for the comparison pairs of choice, was applied. Hochberg's procedure was performed to compare chromosome number distribution for each therapeutic protein across stable and unstable groups, with no pairwise comparison being statistically significant. This indicates that chromosome number distribution does not fluctuate between stability and time point groups, suggesting the selection pressure method and different media composition of producing cell lines confers chromosomal stability at the numerical level.

TABLE 1

| | Cell | Median Chromosome | Modal | 90% CI | |
|---|---|---|---|---|---|
| Protein | Line | Number | Range | range | Stability |
| 2 | 1 | 20 | 13 to 22 | 19 to 21 | Unstable |
| | 2 | 19 | 11 to 23 | 16 to 21 | Unstable |
| | 3 | 20 | 11 to 41 | 18 to 30 | Stable |
| | 4 | 20 | 18 to 38 | 19 to 23 | Unstable |
| | 5 | 34 | 5 to 70 | 17 to 38 | Stable |
| | 6 | 38 | 11 to 43 | 20 to 41 | Stable |
| 3 | 1 | 19 | 13 to 38 | 18 to 35 | Stable |
| | 2 | 19 | 12 to 23 | 18 to 21 | Stable |
| | 3 | 19 | 16 to 38 | 18 to 35 | Unstable |
| | 4 | 19 | 17 to 29 | 18 to 20 | Unstable |
| | 5 | 19 | 14 to 36 | 18 to 30 | Unstable |
| | 6 | 35 | 14 to 41 | 19 to 38 | Stable |
| 5 | 1 | 19 | 11 to 40 | 16 to 20 | Stable |
| | 2 | 19 | 13 to 36 | 18 to 22 | Stable |
| | 3 | 19 | 15 to 37 | 19 to 20 | Stable |
| | 4 | 20 | 13 to 39 | 18 to 22 | Unstable |
| | 5 | 20 | 18 to 23 | 18 to 21 | Unstable |
| | 6 | 38 | 12 to 52 | 23 to 40 | Unstable |

Table 1. Median chromosome numbers of productionally stable and unstable cell lines. Modal range of chromosome numbers and 90% confidence interval chromosome numbers are listed. Modal range shows the full range of chromosomal number of the images analysed. 90% CI range shows the range of chromosome number that applies to 90% of the images analysed.

TABLE 2

| Comparison | Unadjusted p-value | Adjusted p-value |
|---|---|---|
| ‘2’ and Stable vs. ‘2’ and Unstable | 0.0734 | 0.2937 |
| ‘5’ and Stable vs. ‘5’ and Unstable | 0.2357 | 0.4215 |
| ‘3’ and Stable vs. ‘3’ and Unstable | 0.3603 | 0.4215 |
| ‘2’ and Stable vs. ‘5’ and Unstable | 0.4215 | 0.4215 |

Table 2. Data was analysed using a 2-way ANOVA approach with therapeutic protein and stability as factors. Unadjusted p-value represents all pairwise comparisons without adjustment for multiplicity (LSD test). Planned comparisons using Hochberg test of therapeutic protein cell lines within stable and unstable groups forms the adjusted p-value. All pairwise comparisons were insignificant.

To characterise telomere length changes in productionally stable and unstable cell lines, telomere quantification was performed for CHOK1a-GS-KO host telomere analysis. To identify whether telomere length plays a role in production stability, the same 18 cell lines were stained using a TTAGGGn fluorescent probe. Telomere length was calculated for 200 images of each cell line, across early and late time points, to assess whether telomere length fluctuates between stable and unstable cell lines, in addition to time. A least square means (LSM) model was applied to the telomere length data set which considers numerous variables of the data. In contrast to the arithmetic mean, LSM is an average based on a linear model that is adjusted for covariates (e.g. time point, chromosome number, protein etc.), providing a better estimate of the true population mean.

LSM calculation of telomere length, considering stability, early and late time points, across modal chromosome numbers were plotted (data not shown). Protein 2 obtained a larger difference in telomere proportion mean when comparing stable and unstable cell lines, however, 95% confidence limit bars indicate that the differences between the means heavily overlap across the data set. Protein 2 difference observed between the stable and unstable telomere proportion LSM was not shared with protein 3 and 5, indicating the increase in telomere proportion for stable cell lines may only be a protein specific difference.

Overall, patterns in telomere length changes does not appear to be consistent across this panel of cell lines. Protein 2 telomere length proportion decreases from early to late timepoints whereas protein 3 and 5 have mixed profiles (increase and decreases in telomere length) dependent on chromosome number category. The varying profiles across early and late time points identified in the LSM plot is reflected in the insignificance of comparing pooled data into early and late categories (pooled T-test, P=0.58, data not shown), corroborating the notion that there is no difference in telomere length proportion over a prolonged culture period.

To assess whether there is an overall difference in telomere length across stable and unstable cell lines, data was pooled into stable and unstable categories (data not shown). Mean telomere length of unstable cell lines was shown to increase by 0.3% from 2% in the stable cell line category. This difference was found to be highly significant (P=<0.0001), however, the large number of images analysed for each group may have contributed to the increased sensitivity of the statistical test. Additionally, 0.3% may not be a large enough increase to elicit a physiological response.

### Characterising Stable and Unstable Cell Line Karyotypes to Understand the Genomic Mutation Landscape of the Productionally Unstable Phenotype

CHOK1a-GS-KO host, used for therapeutic protein production, has a heterogeneous karyotype that is maintained over a 6-month culturing period. Here, MFISH has been utilised to characterise productionally stable and unstable cell lines across early and late time points to identify any differences or commonalities in genomic instability profiles across the different groups.

### Results

Metaphase chromosomes from a panel of stable and unstable cell lines were harvested and ‘painted’ using MFISH, as previously described. Chromosome populations for each cell line was assessed across early and late time points, using the population determining method.

6 stable and 8 unstable cell lines expressing different therapeutic proteins (P2, P3, P5) were selected based on their pre-determined production stability as assessed in automated mini bioreactors (Ambr 15). Cell lines were thawed and then passaged three times to allow for recovery (>98% viability).

Cell lines with a median chromosome number of 19 to 20 were selected for the proceeding analysis, cell lines with chromosome numbers that are considered ‘aneuploid’ were excluded from analysis as these cell lines are not representative of general cell line population identified here (Table 1) and elsewhere.

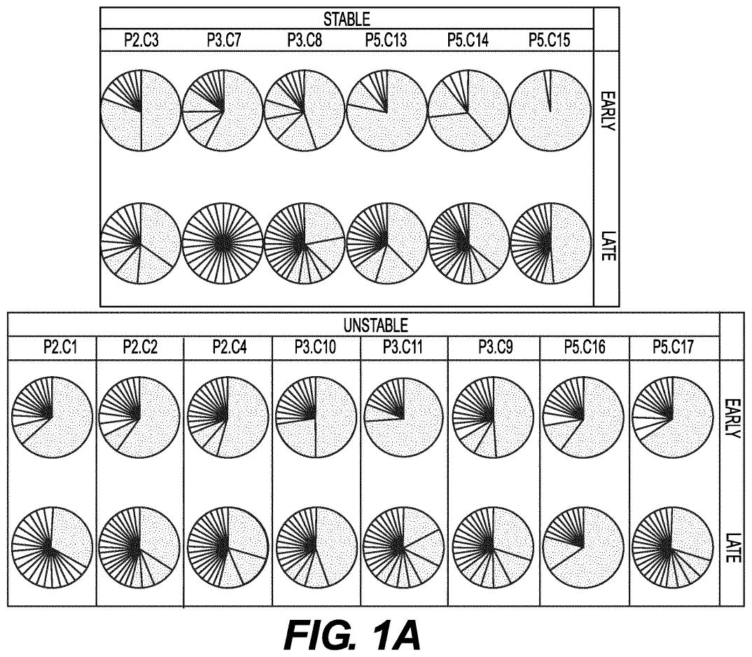

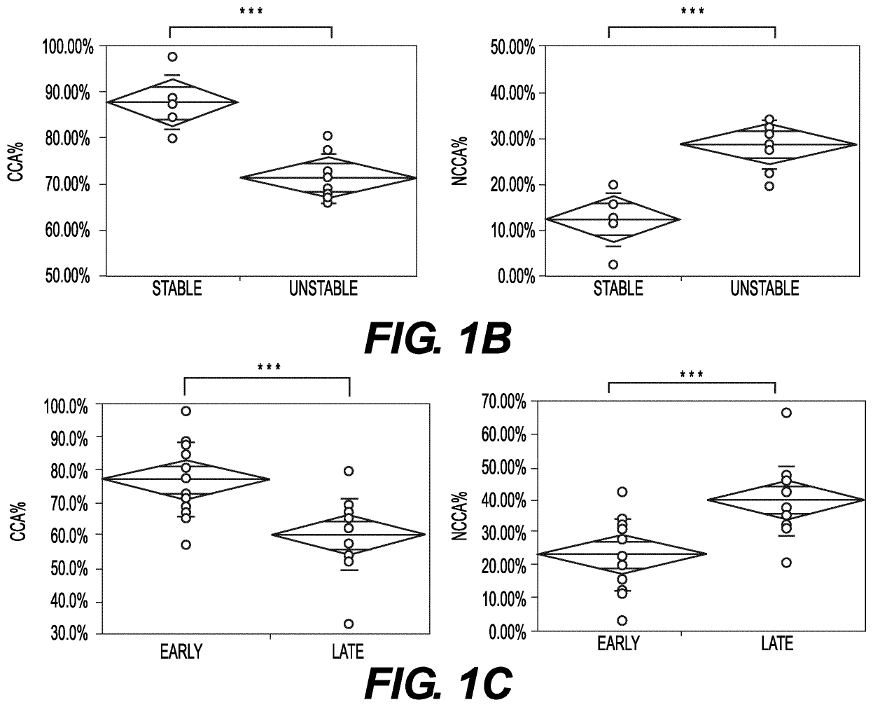

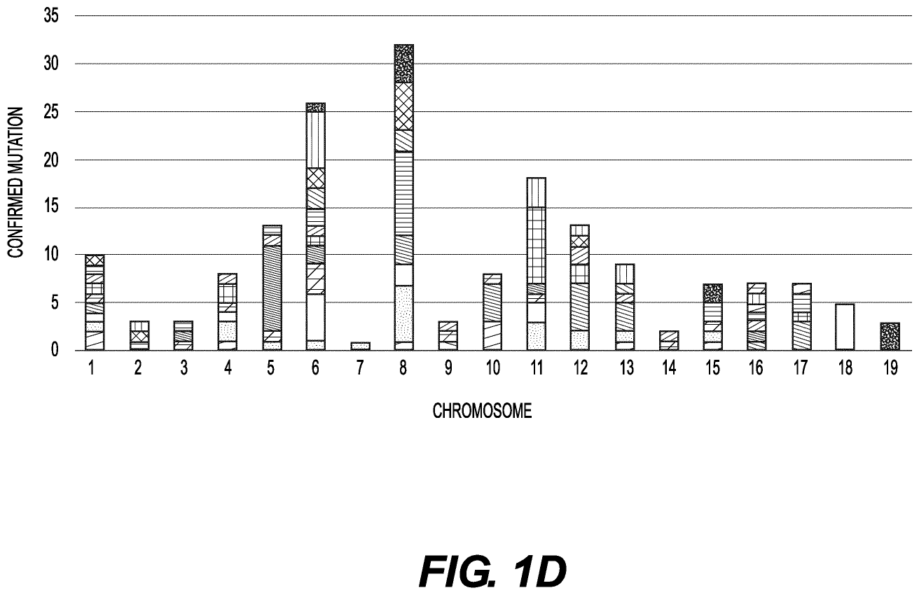

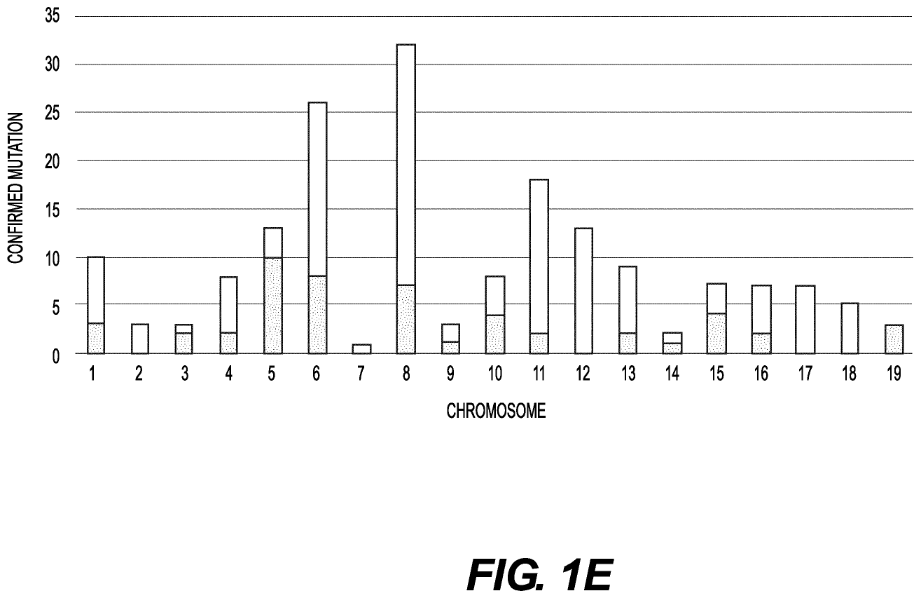

FIG. 1A shows population pie charts of each cell line divided into stability and time point categories. CCA (speckled) and NCCA (plain) pie segments highlight an increase in NCCA populations when comparing stable to unstable and early to late. Overall CCA and NCCA frequencies were calculated across each stability group and differences between each group was statistically significant (Two-way ANOVA, P=0.01). The grand mean was calculated at 78% indicating a potential threshold for production stability designation (FIG. 1B). FIG. 1C shows that CCA and NCCA population frequency difference between early and late time points are statistically significant (Two-way ANOVA, P=<0.0001), indicating that NCCA populations increase over prolonged periods of cell culture, leading to more heterogeneity. The triangles represent the population mean and 95% confidence intervals, blue lines indicate standard deviation. D) Mutations categorised by chromosome; cell lines are represented by the different patterned segments. Chromosome 6 and 8 retain the most mutations with chromosome 6 being mutated in 11 out of 14 cell lines. E) similar bar chart as FIG. 1D except sorted by stability. All chromosomes except 2, 17, 18, and 19 obtained mutations in both stable and unstable cell lines. No pattern of specific chromosome mutations was observed.

Across the panel of stable and unstable cell lines, all obtained multiple karyotypically distinct populations, only differing in the proportion of CCA and NCCA population frequency (FIG. 1a). This indicates that the propensity for gross chromosomal mutations is maintained after transfection of the host and single cell sorting events. Comparisons between the populational composition of stable and unstable groups indicate there is a higher proportion of NCCA populations within the unstable group. Calculating the overall CCA and NCCA percent frequency for stable and unstable categories indicates that a high percentage frequency of CCA populations correlates to productionally stable cell lines (FIG. 1b). Conversely, a greater percentage frequency of NCCA populations was retained in the unstable arm of the cell line panel (FIG. 1b and Table 3 and 4, Two-way ANOVA, P=0.0003). The distinct groupings of % CCA and % NCCA for stable and unstable cell lines indicate that this genomic metric can be utilised as a production stability predictor.

After 6-months of culture, cell lines were re-analysed, and their populations re-determined using the same methodology. Protein 3, cell line 7 (FIG. 1a, P3.C7) late population data was excluded from analysis in FIG. 1c. This is due to the cell line becoming ‘aneuploid’ over the 6-month culturing process and is therefore not comparable with the rest of the data set (data not shown). The distribution of NCCA populations increased drastically regardless of stability except protein 5, cell line 16 (P5.C16) (FIG. 1a). Comparisons of overall CCA and NCCA percent frequency shows a concomitant decrease in CCA and increase in NCCA frequency across the cell culturing period (FIG. 1c and Table 3, Two-way ANOVA, P=<0.0001). Increases in NCCA populations indicates that cell lines become more heterogenous over time, regardless of their production stability (Table 4, P=0.4434). Heterogenous cultures may obtain cells that produce differing amounts of therapeutic protein that may lead to the fluctuations in overall titre over the stability assessment, thus causing the production instability observed within CHO cell lines. In a cell line development environment, this data suggests that a cell line identified as having a prominent level of genetic instability at an early timepoint will become increasingly heterogenous and genetically unstable with time, having a major impact on its ability to homogenously express its therapeutic protein, thereby affecting expression stability.

To understand whether there are common chromosome mutations across cell lines that may be able to identify stability groups, confirmed mutations from the early time point were compiled by chromosome number and coloured by cell line and stability (FIGS. 1d and 1e, respectively). All chromosomes analysed obtained a mutation in one or more cell lines, this indicates that all chromosomes are amenable to deletions, amplifications, rearrangements and/or translocations with no obvious pattern being recognised. Differentiating the mutations by stability indicates that chromosome 6 and 8 have the highest mutation rates overall and the majority of the mutations belong to unstable cell lines. 11 out of 14 cell lines obtained a mutation in chromosome 6, 3 of the 11 are productionally stable and 8 are productionally unstable. 2 out of 3 stable lines express the same therapeutic protein, which may identify a therapeutic protein specific difference with regards to chromosome 6's potential ability to confer production stability in 57% of total cell lines analysed. 5 out of 8 cell lines that obtained a mutation in chromosome 8 are considered productionally unstable, indicating that a mutation in this chromosome could account for 36% of unstable cell lines analysed. Taken together, these results indicate a potential causal-link between production and genomic instability and highlight the prediction power of this method for determining production stability at early time points.

TABLE 3

| | Sums of | Degrees of | Mean | | |
|---|---|---|---|---|---|
| | squares | freedom | square | F-value | p-value |
| Stability | 0.08 | 1 | 0.084 | 10.78 | 0.0033 |
| Timepoint | 0.22 | 1 | 0.222 | 28.71 | 0.0001 |
| Stability * | 0.03 | 1 | 0.025 | 3.26 | 0.0840 |
| Timepoint | | | | | |
| Residuals | 0.18 | 23 | 0.008 | | |

Table 3. ANOVA table of CCA % comparisons between stability and time point. Statistically significant differences in CCA % was obtained in stability (P =< 0.01) and time point (P =< 0.0001).

TABLE 4

| | | | Adjusted |
|---|---|---|---|
| | Comparison | Unadjusted p-value | p-value |
| 1 | Stable Late vs. Stable Early | 7.58516402437071e-05 | 0.0003 |
| 2 | Unstable Early vs. Stable Early | 0.0021817011643801 | 0.0065 |
| 3 | Unstable Late vs. Unstable Early | 0.00667007241142281 | 0.0133 |
| 4 | Unstable Late vs. Stable Late | 0.443449756696863 | 0.4434 |

Table 4. Hochberg's pair-wise comparison adjusted P-values for stability and time point. Significant differences are observed when comparing % CCA between stable and unstable cell lines across early and late time points. There was no significant difference observed between % CCA of late stable and unstable cell lines.

Thus far, a distinct separation of % CCA and % NCCA frequencies between stable and unstable cell line groups have been identified (FIG. 1b). As the cell lines were analysed at an early time point (˜20 generations) the possibility that % CCA vs % NCCA frequency could be utilised as a genomic stability metric, predictive of production stability at an early timepoint, was investigated. This may be beneficial to cell line development timelines as it could provide the means to triage cell lines at a much earlier time point (20 generations) compared to completing the whole stability assessment (70-150+/−10 generations).

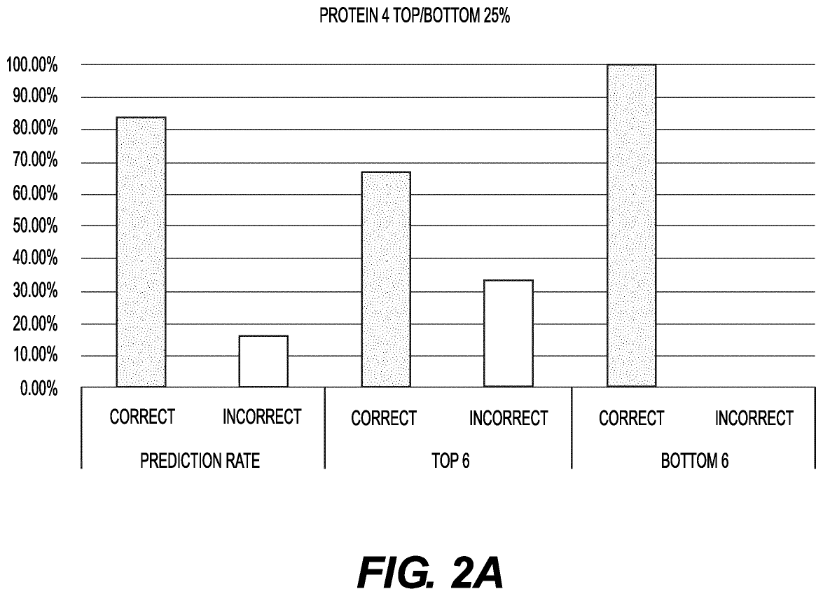

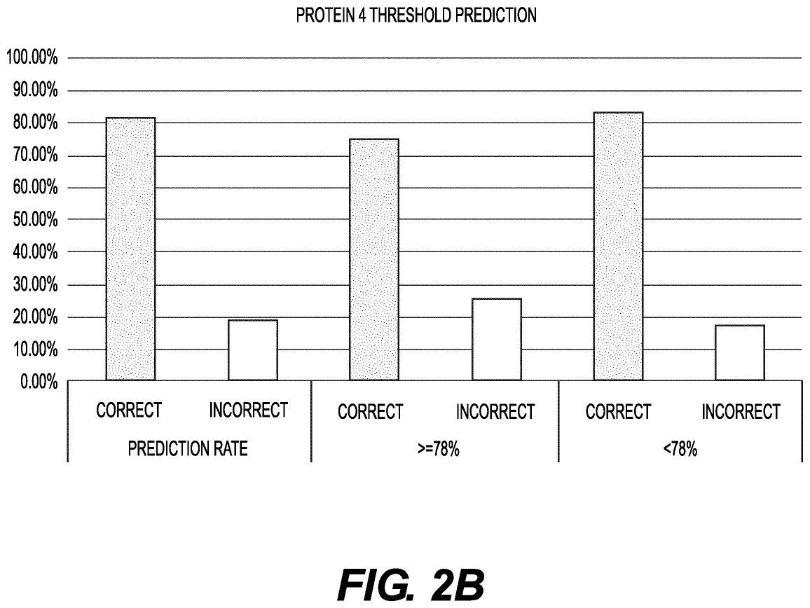

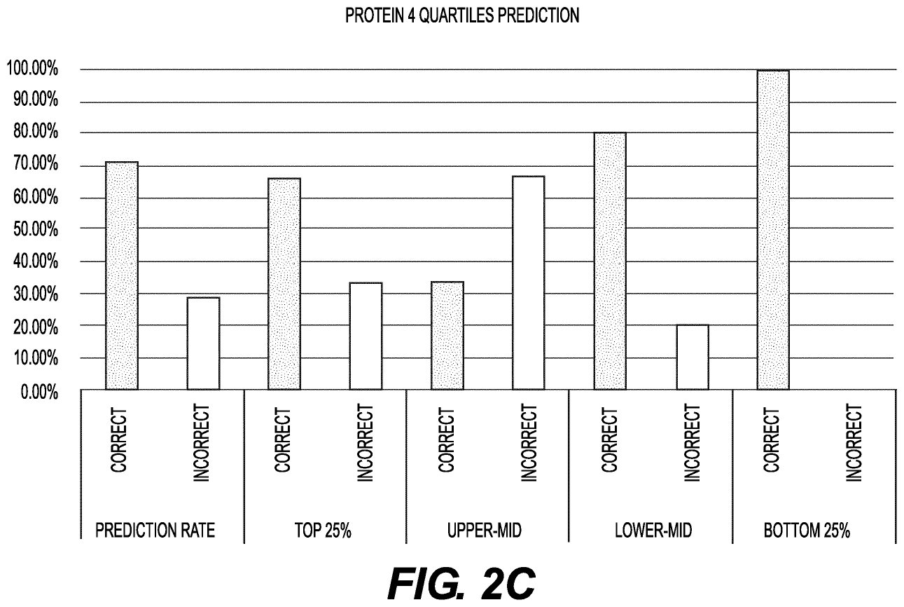

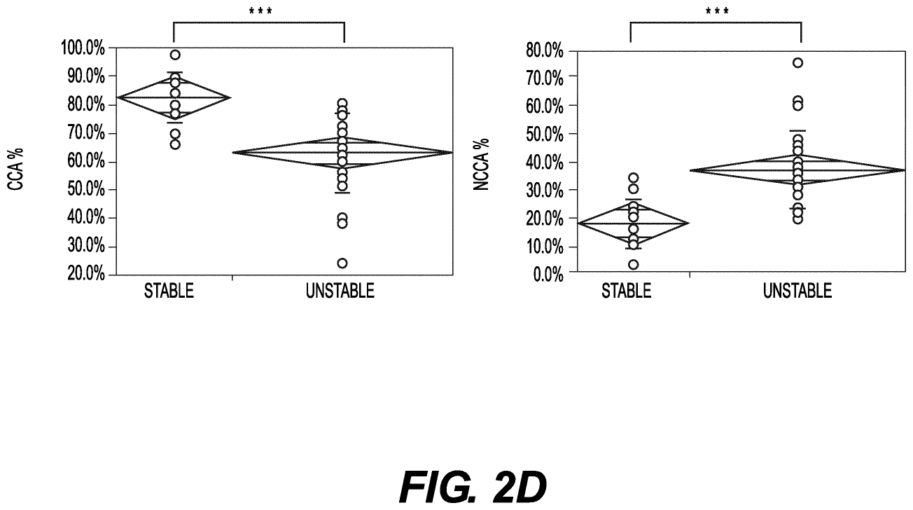

22 cell lines expressing protein 4, were selected to represent a normal distribution of productionally stable and unstable cell lines for any given new live project and their production stability remained blinded until CCA and NCCA populations were analysed. The ability to predict cell line production stability, based on their ranking of % CCA, provides the method's prediction power as it mimics its use in the critical path of cell line development (CLD) for triaging cell lines with unknown production stability. Three separate prediction methods were tested before unblinding the data (FIG. 2).

Top 6 and bottom 6 (FIG. 2a) predictions based on the ranking of % CCA has the potential to quickly identify productionally stable (for cell line progression) and productionally unstable (for triaging) cell lines. Overall, Protein 4 expressing cell lines had a correct prediction rate of 82.5% but this was skewed towards correctly identifying productionally unstable cell lines (100% correct) compared to productionally stable cell lines (67.5% correct).

A second prediction method based on a % CCA threshold, defined from our previous panel of cell lines (78% threshold, FIG. 1b), showed a similar trend in prediction success (FIG. 2b). Anything equal to or above 78% CCA was considered to be productionally stable, less than 78% CCA was considered as unstable. Protein 4 cell lines obtained an overall correct prediction of ˜80% which was more evenly balanced between stable and unstable correct predictions—75% and 82.5%, respectively. A potential benefit of this prediction method is the threshold of % CCA could be better refined as more data is generated, providing a potential increased prediction rate.

Quartile predictions (FIG. 1c) could be utilised to readily identify the top 25% stable cell lines and the bottom 50% productionally unstable lines. Robustly triaging the bottom 50% could drastically increase cell line development capacity by freeing up limited mini bio-reactor space. Protein 4 obtained a 70% correct prediction overall which was largely obtained in the bottom 50% (lower-mid=80% correct, bottom 25%=100% correct) whilst obtaining 67.5% correct prediction rate for the top 25% of stable cell lines.

Overall, predictions using all three prediction methods were successful.

Data presented here indicates that there is a significant difference between the heterogeneity of productionally stable and unstable cell lines, when populations are grouped by CCA and NCCA designations. Interestingly, all cell lines obtain heterogenous karyotypes that is exacerbated over prolonged culture periods, reflecting observations of cell line titre drastically decreasing after ˜100 generations (data not shown). Increases of NCCA populations leads to increased genetic heterogeneity which seems to impact a cell lines ability to maintain production of its therapeutic protein. Conversely, a de novo mutation that is acquired but allows the cell to establish itself within the culture flask (>=5% frequency) appears to be correlated with production stability, as cell lines with heterogenous populations that are predominantly CCA are on the whole productionally stable (FIG. 1a, b and Table 3). The data presented here represents the first study to investigate novel findings on a potential mechanism for cell line production stability in an industry relevant panel of cell lines (40 cell lines across four different therapeutic proteins). Promising production stability prediction results, across multiple therapeutic protein expressing cell lines, provides evidence that the prediction method could be robust enough to be utilised in an industry setting.

### Example 4: Impact of Genetic Stability on Production Stability

Karyotypic heterogeneity of productionally stable and unstable cell lines has thus far been assessed during routine maintenance culture (Example 3). To understand how CHOK1a-GS-KO based cell line heterogeneity fluctuates within a production environment, which is optimised to promote increased production of therapeutic protein, experiments were designed to assess genomic instability during normal production run conditions and in the presence of a DNA damaging agent.

An experiment was designed to assess overall DNA damage effect within a production environment, using Neocarzinostatin as a DNA damaging agent. 6 productionally stable and 6 productionally unstable cells lines were selected from previously analysed cell lines in the initial productionally stable vs unstable and blinded validation panel of cell lines (Example 3). Cell line production cultures were set up in duplicate and contained two groups of non-treated cell lines and treated with 1 ng/ml Neocarzinostatin at day 0 only, using the 24 deep well production run method. Chromosomes were harvested on day 8 of the production run to assess karyotype population heterogeneity. Day 8 was selected as a potential timepoint that would allow the stress of the production environment to elicit any potential effects, whilst maintaining a high enough % VCC (viable cell count) to allow for appropriate sampling for analysis (data not shown).

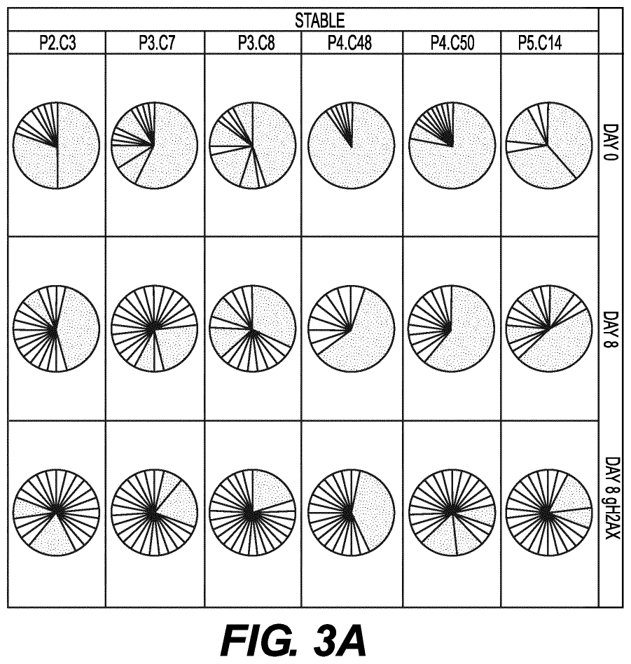

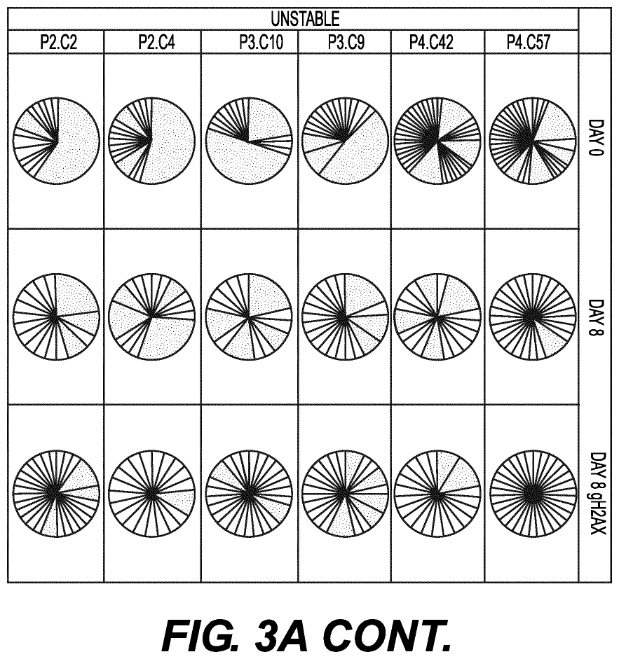

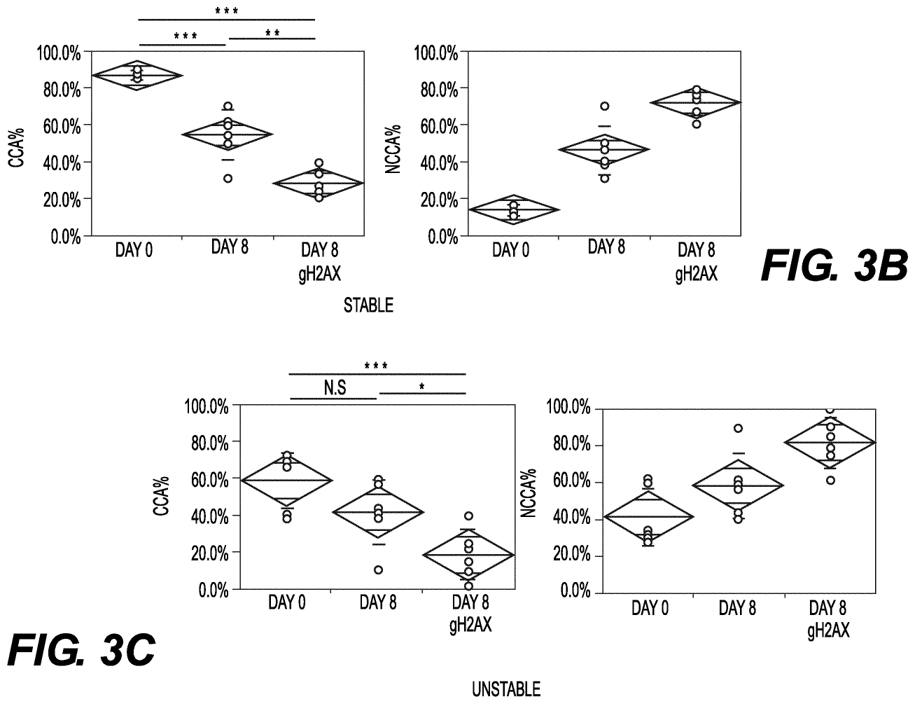

Karyotype heterogeneity was assessed using MFISH as previously described. Karyotype populations were assigned CCA (>5%) or NCCA (=<5%) designations based on their frequency of occurrence. Day 0 represents the baseline karyotypic heterogeneity that the cell line obtained before going through the production run protocol, which is designed to push the cells to produce as much therapeutic protein as possible. As observed in the previous stable and unstable cell line panel, productionally stable cell lines obtained a greater proportion of CCA populations compared to their productionally unstable counter parts by ˜29% (FIGS. 3a, b and c, Table 5 and 6, P=0.004).

After 8 days within the production run environment, % CCA decreased by 32% in stable cell lines and ˜17% in unstable cell lines (FIGS. 3b and c, Tables 5 and 6, P=<0.0001*** and P=0.07n.s, respectively). This suggests the environmental stress of the production run has an impact on genetic stability as there are increases in NCCA populations (˜32% and ˜17%) compared to a less stressful maintenance environment at day 0. The addition of a DNA damaging agent exacerbated NCCA population increase, compared to day 8, by ˜26% for stable cell lines and 23% for unstable cell lines (FIGS. 3b and c, Table 5 and 6, P=0.006 and P=0.014, respectively).

An increase in NCCA populations, upon the addition of a DNA damaging agent, provides evidence that increases in DNA damage within the cell lead to the genomic instability (increase in NCCA populations) witnessed.

TABLE 5

| | Sample | Stability | Mean | Lower 95% CI | Upper 95% CI |
|---|---|---|---|---|---|
| 1 | Day 0 | Stable | 0.866 | 0.760 | 0.971 |
| 2 | Day 8 | Stable | 0.544 | 0.438 | 0.649 |
| 3 | Day 8 gH2AX | Stable | 0.282 | 0.176 | 0.388 |
| 4 | Day 0 | Unstable | 0.588 | 0.482 | 0.694 |
| 5 | Day 8 | Unstable | 0.415 | 0.309 | 0.521 |
| 6 | Day 8 gH2AX | Unstable | 0.184 | 0.078 | 0.290 |

TABLE 6

| | Comparison | Unadjusted p-value | Adjusted p-value |
|---|---|---|---|
| 1 | Day 8 gH2AX Stable vs. Day 0 Stable | 6.72617428421063e-09 | <0.0001 |
| 2 | Day 8 gH2AX Unstable vs. Day 0 Unstable | 5.33622199627715e-06 | <0.0001 |
| 3 | Day 8 Stable vs. Day 0 Stable | 0.000125955500982267 | 0.0009 |
| 4 | Day 0 Unstable vs. Day 0 Stable | 0.000675965734175499 | 0.0041 |
| 5 | Day 8 Stable vs. Day 8 gH2AX Stable | 0.00121681933485585 | 0.0061 |
| 6 | Day 8 Unstable vs. Day 8 gH2AX Unstable | 0.00362759781038302 | 0.0145 |
| 7 | Day 8 Unstable vs. Day 0 Unstable | 0.0246282525808061 | 0.0739 |
| 8 | Day 8 Unstable vs. Day 8 Stable | 0.089086173693866 | 0.1782 |
| 9 | Day 8 gH2AX Unstable vs. Day 8 gH2AX Stable | 0.190188840671827 | 0.1902 |

### Example 5: Data Analysis Workflow

Cell characterisation and analysis should be industrially scalable, and data rapidly generated to provide a greater depth of host cell characterisation, without impacting project timelines during cell line development. Primarily, image analysis and liquid handling for genetic screens were identified as major bottlenecks for these types of analyses. Solutions conceptualised and implemented to allow for industrialisation of image analysis are outlined.

Image analysis is often performed using software that allows characterisation of fluorescent images, but often in a manual and subjective manner (e.g. ImageJ). In a bid to remove this subjectivity from analyses and decrease analysis time lines, image analysis workflows were created on CellProfiler™ (http://cellprofiler.org/) using their built-in image analysis modules to confirm mutations observed. Described herein are said workflows and how they could be applied on the CLD critical path.

Fluorescent based image analysis represents an important tool for cellular characterisation. It provides the ability to visualise any protein or DNA sequence (when appropriate antibodies and probes are available) within the cell that aids a better description of the underlying cellular biology when investigating a desired phenotype. However, image analyses have historically been analysed manually, opening up the analyses to unintentional bias and subjectivity that may impact the output of results.

### Results

In the above Examples, analysis of MFISH karyotypes of CHOK1a-GS-KO host, productionally stable and unstable cell lines, was performed manually. To remove potential subjectivity and bias in mutation identification, a CellProfilerm workflow was created to extract the fluorescent intensities from 5 separate colour channels, from each individual chromosome. Single channel images are extracted from the Metafer software (Metasystems, V5.7.4) and undergo a series of threshold corrections to remove background fluorescence.

Chromosome masks are identified through the identify primary objects module using the DAPI channel. Automated masks are manually edited to remove any artefacts (e.g. cells or debris) within the image. Additionally, chromosomes that are in close proximity can be split into individual masks to faithfully replicate the original image. Semi-automated chromosome segmentation allows for the extraction of fluorescent intensity values of pixels in each colour channel contained within the mask.

Expressing the fluorescent pixel intensities from each channel within a single chromosome mask, as a percentage of each other, provides a chromosome colour profile (data not shown) that is utilised to confirm chromosome mutations that are visually identified. This allows the analyst to have a colour profile of the mutation in question providing further evidence that the mutation observed by the analyst is reflected at the fluorescent pixel intensity level. Chromosomes are ‘painted’ using a colour coding system that is built into the proprietary Metasystems software.

Although the semi-automated CellProfiler™ workflow provides an objective means to profile a chromosomal mutation observed during MFISH karyotype analysis, the workflow can still be laborious due to manual editing of each individual image and post analysis processing of fluorescent intensity data. With the current rise in interest in artificial intelligence and machine learning (AI/ML), AI/ML-based methods were used to fully automate the end-to-end process from MFISH images to stability prediction, removing subjectivity, enhancing reproducibility and reducing overall analysis timelines. The end-to-end automated data analysis pipeline is described in Example 6.

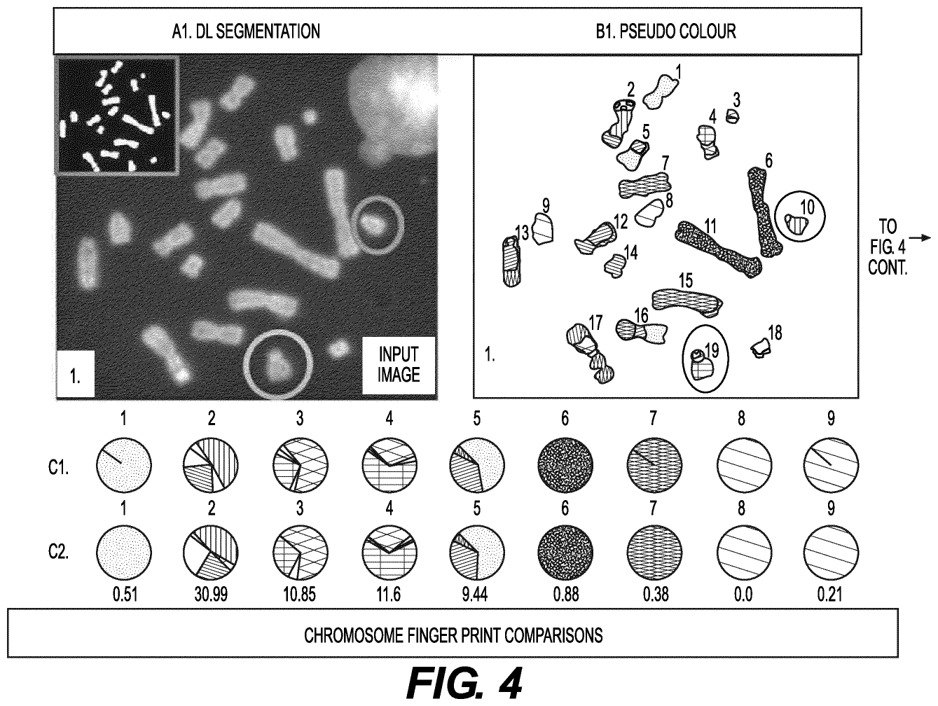

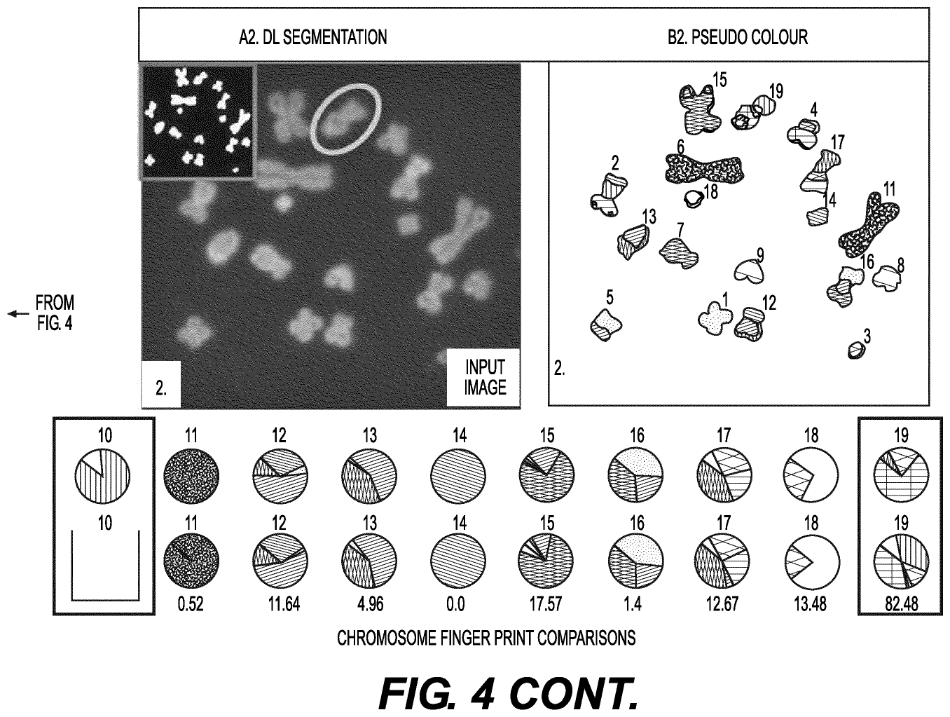

An example of automated mutation detection is described in FIG. 4. Chromosomes assigned number 10 and 19 are shown to be separate within image 1 (a1 and b1, circled). Within image 2, these chromosomes have undergone a translocation event, which can be confirmed using DAPI channel and pseudo coloured image (a2 and b2, circled). Upon performing the pairwise linear assignment (C1=image 1 and C2=image 2), no match can be found for chromosome 10 (as it is not present in image 2) and chromosome 19 has been matched to the mutated chromosome, however with a large matching cost of 82.48. To put this value into context, two chromosomes with genetic similarity (number 6) has a matching cost of 0.88. Therefore, a matching cost threshold can be applied to quickly identify mutations in large image sets (e.g. >50 matching cost=a mutation).

To validate the end-to-end automated data analysis pipeline (referred to as APW), images used in manual MFISH analyses were analysed through the APW algorithm and data was compared against manual methods. APW identification of CCA and NCCA populations were largely consistent compared to manual method (FIG. 5a). Unstable cell lines obtained a larger proportion of NCCA populations compared to their stable counterparts, as observed with manual analysis. Comparing CCA and NCCA frequency showed a significant difference between stable and unstable groups, as observed in manual analyses (FIG. 5b, pooled T-test, P=<0.05). Upon comparing CCA % and variance of average matching cost, it was observed there was a distinct separation between stable and unstable cell lines based on their matching cost variance, indicating variance of average matching cost distribution could be used as another genomic instability metric analogous to CCA and NCCA designated populations (FIG. 5c).

Performing the manual MFISH karyotyping 40 images per cell line for 48 cell lines culminates in a total analysis time (minus sample preparations) of 159 hours. In contrast to APW that can complete the same analysis in 1.3 hours, a time saving of ˜157 hours for researchers.

Due to the highly laborious nature of the manual analysis, 40 images per sample were analysed. APW analysis time savings provides the means to increase images analysed from 40 to 200-400 images per cell line, providing a greater in-depth characterisation of the cell culture flask. APW provides an upscaled (200 images per cell line, 48 cell lines) analysis time saving of 32.9 days, providing an industrialised algorithm that could be integrated into CLD's critical path, without impacting project timelines.

Upon integration into CLD's critical path, APW will be utilised as an early cell line triaging method. A standard stability assessment requires 48 cell lines, belonging to a single therapeutic protein, which is cultured from 4 to 6 months before the cell lines production stability is identified. Through performing a stability prediction on a blinded panel of cell lines, it was observed that the prediction workflow obtained greater correct prediction results for unstable cell lines. Using this method to triage unstable cell lines would provide an enrichment of stable cell lines after one month, reducing the number of cell lines that are subjected to the full stability assessment to 12 cell lines per therapeutic protein. Therefore, four therapeutic proteins could have their stability assessed in a single stability run, in a 7-month period. In the current general sequential format (1 therapeutic protein, 48 cell lines, 4-6 months per protein), it would take 16 months to assess four therapeutic protein cell lines stabilities. Thus, implementing APW could lead to a 4-fold increase in CLD capacity and savings on CMC timelines.

### Example 6: End-to-End Automated Data Analysis Pipeline (Referred to as APW)

### End-to-End Automated Data Analysis Pipeline Devised to Streamline MFISH Production Stability Prediction Timelines and Provide Industry Scalable Data Analysis Tools

The rationale behind assessing genomic instability with MFISH is to obtain early predictors of clonal cell line production instability and triage out unwanted clonal cell lines at an earlier time point to reduce cycle time and free up additional resource. To realise the value of MFISH, automated image analysis pipelines are needed to avoid the time and resources required by visual inspection of images and manual data processing. Additional benefits of an automated image analysis pipeline over manual are objectivity and reproducibility.

### Results

To enable use of MFISH in a production setting, an end-to-end automated image analysis pipeline was designed to predict cell line production stability/instability from a set of MFISH images.

Each MFISH image is a 6-channel TIFF where channel 1 is the DAPI channel used for segmentation and the remaining 5 channels (2, . . . , 6) are used to determine the pixel pseudo-colours from a palette of 12 colours.

The analysis pipeline is comprised of five stages which can be described for a set of MFISH images of a given cell line as follows:
1. Segment Chromosomes: For every pixel in every image, classify the pixel as 1 if it belongs to a chromosome, otherwise 0.
2. Describe Chromosomes: For every chromosome pixel in every image, assign a pseudo-colour label from 1 to 12 and describe every chromosome in every image by a 12-sector pie whose i-th sector corresponds to pseudo-colour i and the size of sector i is the proportion of the chromosome pixels of colour i.
3. Match Chromosomes: For every pair of images, determine a one-to-one correspondence, and associated average matching cost per chromosome, between chromosomes of the first image and chromosomes of the second image.
4. Compute Genomic Stability Biomarker: Calculate the variance of the average matching cost distribution.
5. Predict Protein-Production Stability: Apply a pre-determined threshold to the variance to classify the cell line as either protein production stable or unstable.

### Segmenting Chromosomes

Images were segmented using U-Net which is a convolutional neural network that was designed to segment cell nuclei on few training images. The architecture is a feed-forward network consisting of repeating layers of contraction via a convolution, a rectified linear unit and a max-pooling layer, followed by repeating layers of expansion via a deconvolution layer and an up-sampling layer. Contracting and expanding layers are also connected through concatenation which gives the architecture its U shape.

There were several challenges to segmenting chromosomes that required modifications to the standard training and deployment of the U-Net. The first modification was to the binary, cross-entropy loss function so that misclassification of pixels at the boundaries of chromosomes in close proximity is heavily penalised. The loss function was multiplied by a weight matrix whose ij-th entry was high if the pixel at the ij-th position in the image was between chromosomes in close proximity. The second modification was to overcome the presence of image artefacts and to filter out other non-chromosome cellular structures. Two U-Net models were trained. The first was to predict foreground pixels (i.e. those belonging to chromosome) whereas the second was to predict background pixels. The two sets of pixel classifications were combined via intersection to arrive at a final segmentation.

### Describing Chromosomes

Chromosomes were coloured using a Gaussian mixture model that was trained on an image set from a single cell line. The pixels classified as belonging to chromosomes can be considered as points in 5-dimensional space colour space, where dimension i corresponds to the greyscale intensity of the pixels in the i-th colour channel. The position of the pixel in colour space determines its pseudo-colour. Gaussian mixture models are probabilistic models that can be used for clustering data points into subpopulations. To build the pseudo-colouring model, images from a single cell line were first segmented then their chromosome pixels were assigned to 12 pseudo-colour populations, by a Gaussian mixture model, based on their coordinates in colour space. This model was then applied to every segmented image of every remaining cell line. The results were compared to those generated using the Metabase software.

Segmented and pseudo-coloured chromosomes can be characterised by their pseudo-colour proportions to facilitate comparison with chromosomes across a single cell line. More specifically, each chromosome is assigned a 12-tuple fingerprint whose i-th component is the percentage of the chromosome of pseudo-colour i. Such a chromosome fingerprint can be represented visually by a pie chart whose i-th sector is coloured by pseudo-colour i and sized by the i-th component of the fingerprint.

### Matching Chromosomes

Given a pair of segmented and pseudo-coloured MFISH images, the task was to identify a set of one-to-one correspondences between the chromosomes of image 1 and the chromosomes of image 2 such that chromosomes with similar pseudo-colour patterns are matched together. This matching was a necessary step to enable comparison between the chromosomal populations imaged across an entire cell line. The degree of matching can be calculated with a cost function that quantifies the pseudo-colour discordance between a pair of chromosomes. The set of correspondences was determined by solving the linear assignment problem with cost matrix C whose rows and columns are indexed by the chromosomes of images 1 and 2, respectively, and whose ij-th entry is the cost of matching chromosome i from image 1 with chromosome j from image 2.

The output of the Hungarian algorithm that was used to solve the linear assignment problem was a set of one-to-one correspondences between the imaged chromosomal populations that minimises the total matching cost. By design, matched chromosomes tend to yield a low matching cost and have a similar pseudo-colour fingerprint. Should the populations differ at all due to a chromosomal aberration, the total matching cost will be higher than the expected value. To account for variation in numbers of chromosomes in a population, the total matching cost was averaged over the number of chromosomes. This average, along with all other averages calculated over every unique pair of images in the image set, form an average matching cost distribution. An example of the output from this phase of the algorithm is depicted in FIG. 4. Note that an image of 19 chromosomes is matched with an image of 18 chromosomes which results in one chromosome not being assigned a match. As can be seen from FIG. 4, there is no cost optimal match for chromosome 10 in image one, so this chromosome is not paired. Note also that chromosome 19 in image 1 is paired with chromosome 19 in image 2 with an outlier matching cost of 82%. Chromosome 19 in image 2 is a fusion of chromosomes 10 and 19 in image 1. This chromosomal aberration is reflected in the statistically high matching cost for chromosome 19 in images 1 and 2.

### Computing a Genomic Stability Metric

A metric for genomic instability is the variance of the average matching cost distribution for the cell line. A high variance is indicative of a high degree of genomic instability, whereas a low variance suggests a cell line is genomically stable. This observation is confirmed by the correlation between the variance and the manually-derived % CCA as shown in FIG. 5, scatter plot c).

### Predicting Protein Production Stability

To predict protein production stability on new cell lines, an appropriate threshold must be estimated from existing average matching cost distribution variance and subsequently applied to variance derived from new cell lines. The 14 cell lines analysed to date have known protein production stability outcomes and the FIG. 5, c) scatter plot of automated-calculation of variance versus manually-derived % CCA, where each point corresponds to cell line that is speckled if stable protein production and plain if unstable protein production, shows a clear separation between the two protein production stability classes. To identify an appropriate variation threshold above which, cell lines will be predicted as protein production unstable, otherwise stable, a decision tree was built using the existing 14 cell lines, although this is not strictly necessary. Assuming no changes to the experimental protocol, this threshold can be applied to new data to predict cell line protein production stability.

It is worth noting that should any modifications be made to the experimental protocol, all machine learning models deployed in the workflow will require retraining on the new data. Explicitly, this means rebuilding the segmentation model, the pseudo-colouring model and finally the decision tree model.

### Example 7: Conclusion

Results presented in this application have provided characterisation of the interrelation between genomic and production instability within CHOK1a-GS-KO host and CHOK1a-GS-KO based producer cell lines. Previous work (Vcelar et al., 2018a; Vcelar et al., 2018b) has provided genomic instability characterisation of CHOK1 based host cell lines during routine maintenance and have tracked genomic heterogeneity over the single cell cloning process in a variety of cell culture conditions. Within these previous studies, no attempt was made to elucidate a causative pathway of the heterogeneity observed.

In line with observations by Vcelar et al., (Vcelar et al., 2018a; Vcelar et al., 2018b), vast heterogeneity both within CHOK1a-GS-KO host and CHOK1a-GS-KO producing cell lines was observed, regardless of stability. The studies disclosed herein have expanded on these findings further by applying clonal (CCA) and non-clonal (NCCA) chromosomal aberration designations, used within the cytogenetic field for disease diagnosis, providing a general mutation metric that describes the overall mutation landscape within the cell culture flask. Although a stable cell line may have multiple populations, it is the ratio of CCA (genetically stable mutation) or NCCA (genetically unstable/rare) that defines the overall genomic stability of the cell line.

Applying this metric, the inventors have established a correlation between increased mutations (high % NCCA) and production instability, which showed a consistent trend in the cell lines expressing 4 therapeutic proteins. Moreover, the inventors have shown that this metric could be used for production stability prediction at an early time point, testing the methodology on a blinded panel of cell lines to recapitulate its use on a live CLD project. This study is the first to test novel findings in an industry relevant panel of cell lines (36 cell lines across 4 therapeutic proteins) that produce full sized therapeutic proteins.

The inventors have further shown that variance of average matching cost is also analogous to % CCA, and as such, that variance of average matching cost may also be used as a further genomic instability metric. Further, by virtue of the mathematical relationship between variance and SD, SD of average matching cost can also be used as a genomic instability metric.

Automation of the manual MFISH based stability prediction method, allowed rapid objective analysis of samples with results that correlate well with manual results. This provides a fully scalable method that allows greater characterisation (increased number of cells analysed) and rapid analysis to provide output results within an industry time frame.

Overall, the results disclosed in the present application provides a method that tracks mutations and shows % CCA or % NCCA, variance of average matching cost distribution or SD of average matching cost distribution as a viable genomic stability metric that can be utilised for production stability prediction.

### REFERENCES

Berk, A. J. (2005). Recent lessons in gene expression, cell cycle control, and cell biology from adenovirus. Oncogene 24, 7673-7685.
de Lange, T. (2002). Protection of mammalian telomeres. Oncogene 21, 532-540.
Gisselsson, D., Pettersson, L., Hoglund, M., Heidenblad, M., Gorunova, L., Wiegant, J., Mertens, F.,
Dal Cin, P., Mitelman, F., and Mandahl, N. (2000). Chromosomal breakage-fusion-bridge events cause genetic intratumor heterogeneity. Proc Natl Acad Sci USA 97, 5357-5362.
Hayflick, L. (1965). The Limited in Vitro Lifetime of Human Diploid Cell Strains. Exp Cell Res 37, 614-636.
Hayflick, L., and Moorhead, P. S. (1961). The serial cultivation of human diploid cell strains. Exp Cell Res 25, 585-621.
Huen, M. S., and Chen, J. (2008). The DNA damage response pathways: at the crossroad of protein modifications. Cell Res 18, 8-16.
Marotta, M., Chen, X., Watanabe, T., Faber, P. W., Diede, S. J., Tapscott, S., Tubbs, R., Kondratova, A., Stephens, R., and Tanaka, H. (2013). Homology-mediated end-capping as a primary step of sister chromatid fusion in the breakage-fusion-bridge cycles. Nucleic Acids Res 41, 9732-9740.
O'Sullivan, R. J., and Karlseder, J. (2010). Telomeres: protecting chromosomes against genome instability. Nat Rev Mol Cell Biol 11, 171-181.
Roos, W. P., and Kaina, B. (2006). DNA damage-induced cell death by apoptosis. Trends Mol Med 12, 440-450.
Schmutz, I., and de Lange, T. (2016). Shelterin. Curr Biol 26, R397-399.
Sha, J., Ghosh, M. K., Zhang, K., and Harter, M. L. (2010). E1A interacts with two opposing transcriptional pathways to induce quiescent cells into S phase. J Virol 84, 4050-4059.
Thomas, R., Marks, D. H., Chin, Y., and Benezra, R. (2018). Whole chromosome loss and associated breakage-fusion-bridge cycles transform mouse tetraploid cells. EMBO J 37, 201-218.
Vcelar, S., Jadhav, V., Melcher, M., Auer, N., Hrdina, A., Sagmeister, R., Heffner, K., Puklowski, A., Betenbaugh, M., Wenger, T., et al. (2018a). Karyotype variation of CHO host cell lines over time in culture characterized by chromosome counting and chromosome painting. Biotechnol Bioeng 115, 165-173.
Vcelar, S., Melcher, M., Auer, N., Hrdina, A., Puklowski, A., Leisch, F., Jadhav, V., Wenger, T., Baumann, M., and Borth, N. (2018b). Changes in Chromosome Counts and Patterns in CHO Cell Lines upon Generation of Recombinant Cell Lines and Subcloning. Biotechnol J 13, e1700495.
Kremkow, B. G., Baik, J. Y., MacDonald, M. L., and Lee, K. H. (2015). CHOgenome.org 2.0: Genome resources and website updates. Biotechnol J 10, 931-938.
Yusufi, F. N. K., Lakshmanan, M., Ho, Y. S., Loo, B. L. W., Ariyaratne, P., Yang, Y., Ng, S. K., Tan, T. R. M., Yeo, H. C., Lim, H. L., et al. (2017). Mammalian Systems Biotechnology Reveals Global Cellular Adaptations in a Recombinant CHO Cell Line. Cell Syst 4, 530-542 e536.
Deaven, L. L., and Petersen, D. F. (1973). The chromosomes of CHO, an aneuploid Chinese hamster cell line: G-band, C-band, and autoradiographic analyses. Chromosoma 41, 129-144.
Wurm, F. M. (2004). Production of recombinant protein therapeutics in cultivated mammalian cells. Nat Biotechnol 22, 1393-1398.
Butler, M. & Spearman, M. The choice of mammalian cell host and possibilities for glycosylation engineering. Curr Opin Biotechnol 30, 107-112, doi:10.1016/j.copbio.2014.06.010 (2014).
Walsh, G. Biopharmaceutical benchmarks 2018. Nat Biotechnol 36, 1136-1145, doi:10.1038/nbt.4305 (2018).
Derouazi, M., Martinet, D., Besuchet Schmutz, N., Flaction, R., Wicht, M., Bertschinger, M., Hacker, D. L., Beckmann, J. S., and Wurm, F. M. (2006). Genetic characterization of CHO production host DG44 and derivative recombinant cell lines. Biochem Biophys Res Commun 340, 1069-1077.
Heng et al. Molecular Cytogenetics (2016) 9:15

1. A method of predicting production stability and/or production instability of a clonal cell line, the method comprising the steps of
(a) growing two or more clonal cell lines in separate cell cultures
(b) karyotyping the cells in each cell culture; and
(c) deriving a genomic instability value from the karyotyping of step (b).

2. A method of selecting a cell line which expresses a therapeutic protein, the method comprising the steps of
(a) growing two or more clonal cell lines in separate cell cultures
(b) karyotyping the cells in each cell culture
(c) deriving a genomic instability value from the karyotyping of step (b); and
(d) selecting a clonal cell line based on the genomic instability value of step (c).

3. A method of selecting a high-titre producing clonal cell line for large scale therapeutic protein production, the method comprising the steps of
(a) growing two or more clonal cell lines in separate cell cultures
(b) karyotyping the cells in each cell culture
(c) deriving a genomic instability value from the karyotyping of step (b); and
(d) selecting a clonal cell line based on the genomic instability value of step (c).

4. The method of according to claim 2, wherein karyotyping comprises identifying chromosomal aberrations of the clonal cell lines.

5. The method according to claim 2, wherein karyotyping comprises performing multi-colour fluorescence in situ hybridisation (MFISH), spectral karyotyping (SKY) or Giesma banding (G banding).

6. The method according to claim 2, further comprising after step (b), the step of determining subpopulations of each cell culture by karyotype.

7. The method according to claim 6, wherein deriving the genomic instability value comprises assigning each subpopulation as comprising clonal chromosomal aberration (CCA) or non-clonal chromosomal aberration (NCCA).

8. The method according to claim 7, wherein deriving the genomic value further comprises the step of determining a percentage CCA and/or percentage NCCA for each clonal cell line.

9. The method according to claim 2, wherein deriving the genomic instability value comprises determining an average matching cost distribution.

10. The method according to claim 9, wherein deriving the genomic instability value comprises determining a variance of the average matching cost distribution.

11. The method according to claim 2, wherein the genomic instability values are used to i) rank the clonal cells by % CCA or variance of the average matching cost distribution; (ii) derive a % CCA threshold or variance of the average matching cost distribution threshold; and (iii) derive a quartile threshold.

12. The method according to claim 11, wherein the genomic instability values are used to derive a % CCA threshold, optionally wherein the % CCA threshold is at least 70%, further optionally wherein the % CCA threshold is 78%.

13. The method according to claim 2, wherein the step of karyotyping the cells in each cell culture and/or the step of deriving a genomic instability value from the karyotyping is/are automated.

14. The method according to claim 13, wherein automation is computer-implemented automation.

15. The method according to claim 2, wherein the step of karyotyping the cells in each cell culture is carried out between 10 generations and 40 generations, optionally wherein the step of karyotyping the cells in each cell culture is carried out after 10, 15 or 20 generations.

16. The method according to claim 2, wherein the clonal cell line is a mammalian cell line.

17. The method according to claim 16, wherein the mammalian cell line is a Chinese Hamster Ovary (CHO) cell line.

18. The method according to claim 17, wherein the CHO cell line is CHO-K1.

19. The method according to claim 17, wherein the CHO cell line is a glutamine synthetase (GS) knocked out cell.
